(19) United States
(12) Patent Application Publication
(10) Pub. No.: US 2026/0265176 A1
(43) Pub. Date: Sep. 10, 2026

(54) SUBSTITUTED CYCLOHEXENE AND CYCLOHEXANE INHIBITORS OF ORNITHINE AMINOTRANSFERASE

(71) Applicant: Northwestern University, Evanston, IL (US)

(72) Inventors: Wei Zhu, Evanston, IL (US); Allison Nicole Devitt, Evanston, IL (US); Richard Bruce Silverman, Evanston, IL (US)

(21) Appl. No.: 19/166,354

(22) PCT Filed: Mar. 18, 2024
(86) PCT No.: PCT/US2024/020391

§ 371 Date: Sep. 17, 2025

Related U.S. Application Data
(60) Provisional application No. 63/490,934, filed on Mar. 17, 2023.

(51) Int. Cl. C07C 229/48; A61K 31/196

(52) U.S. Cl. CPC C07C 229/48; A61K 31/196; C07C 2601/14; C07C 2601/16

(57) ABSTRACT

Disclosed are amino, halo-substituted cyclohexene or cyclohexane carboxylic acid compounds. The disclosed compounds and compositions thereof may be utilized in methods for modulating ornithine aminotransferase (OAT) activity, including methods for treating diseases or disorders associated with OAT activity or expression such as cell proliferative diseases and disorders.

### CROSS-REFERENCE TO RELATED APPLICATIONS

The present application claims priority to U.S. Provisional Patent Application No. 63/490,934, filed Mar. 17, 2023, the entire contents of which are hereby incorporated by reference.

### STATEMENT REGARDING FEDERALLY SPONSORED RESEARCH

This invention was made with government support under grant number CA260250 awarded by the National Institutes of Health. The government has certain rights in the invention.

### BACKGROUND OF THE INVENTION

Liver cancer is the sixth most common cancer and the second leading cause of cancer-related deaths. Among all primary liver cancers, hepatocellular carcinoma (HCC) is the most common neoplasm, accounting for approximately 85-90% of cases. To date, only four small molecules are available on the market as treatment options for HCC, while they lack target selectivity, thereby resulting in various off-target effects and drug resistance.

The human ornithine d-aminotransferase (hOAT) gene was found to be overexpressed in liver cancer tissues, while pharmacological inhibition of hOAT leads to promising tumor growth reduction. As such, there is a need for hOAT inhibitors that may provide novel therapeutic approaches for treatment of liver cancers, including HCC.

### BRIEF SUMMARY OF THE INVENTION

Disclosed are compounds, compositions and related methods of use for the selective inhibition of ornithine aminotransferase. The disclosed compounds, compositions, and methods can be utilized to treat diseases and disorders associated with ornithine aminotransferase activity.

The disclosed compounds may be described as substituted cyclohexene or cyclohexane compounds. In particular, the disclosed compounds may be described as amino, halo-substituted cyclohexene or cyclohexane carboxylic acid compounds. The disclosed compounds and compositions thereof may be utilized in methods for modulating ornithine aminotransferase (OAT) activity, including methods for treating diseases or disorders associated with OAT activity or expression such as cell proliferative diseases and disorders.

The disclosed compounds may be directed to a compound of the following formula or a dissociated form, a non-protonated form, a zwitterion form, or a salt thereof:

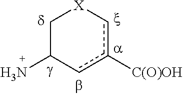

wherein a double bond is present between the α and ζ carbons, or between the α and β carbons, or not present between the α and ζ carbons and between the α and β carbons:

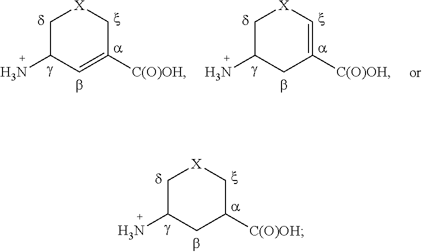

wherein X is CR¹R² or C═CR¹R², and R¹ and R² are independently hydrogen or halo (e.g., a halo such as F, Cl, Br, and I), providing at least one of R¹ and R² is halo.

As indicated, the disclosed compounds may be protonated, for example to form an ammonium moiety, optionally where the compound is present as a salt. The disclosed compounds also may be non-protonated and/or dissociated, for example, where the carboxylic acid moiety is dissociated to form a carboxylate moiety, optionally where the compound is present as a salt. The disclosed compounds may be in zwitterionic form where the compound comprises a protonated ammonium moiety and a dissociated carboxylate moiety, optionally where the compound is present as a salt.

The compounds disclosed herein are without stereochemical or configurational limitation and encompass all stereochemical or configurational isomers, unless stereochemical or configurational limitations are indicated. As illustrated and discussed below, such compounds and/or their intermediates are available as single enantiomers, racemic mixtures from which isomers can be resolved, or diastereomers from which the corresponding enantiomers can be separated. Accordingly, any stereocenter can be (S) or (R) with respect to any other stereocenter(s). As another separate consideration, various compounds can be present as an acid or base salt, either partially or fully protonated, for example at the amino group to form an ammonium moiety, and/or either partially or fully dissociated, for example at the carboxyl group to form a carboxylate substituent or moiety. In certain such embodiments, with respect to an ammonium substituent or moiety, the counter ion can be a conjugate base of a protic acid. In certain such or other embodiments, with respect to a carboxylate substituent or moiety, the counter ion can be an alkaline, alkaline-earth or ammonium cation. Further, it will be understood by those skilled in the art that any one or more the compounds disclosed herein can be provided as part of a pharmaceutical composition comprising a pharmaceutically acceptable carrier component for use in conjunction with a treatment method or medicament.

The disclosed compounds and compositions may be utilized in methods for modulating ornithine aminotransferase (OAT) activity. Such methods can comprise providing a compound as disclosed herein, or a pharmaceutical composition comprising the compound as disclosed herein, and contacting a medium comprising an OAT with the compound or the pharmaceutical composition.

In certain embodiments, the disclosed methods may be directed to reducing activity of an OAT expressed by a cancer, which may include but is not limited to hepatocellular cancer (HCC), or other cancers that express or overexpress OAT. Such a method can comprise providing a compound as disclosed herein, or a pharmaceutical composition comprising the compound as disclosed herein, and contacting the cancer cells with the compound or the pharmaceutical composition.

In certain embodiments, the disclosed methods may be directed to treating a cell proliferative disease or disorder in a subject in need thereof. Suitable cell proliferative diseases and disorders may include cancers that express or overexpress OAT such as, but not limited to, hepatocellular carcinoma (HCC). Such a method can comprise administering to such a subject in need thereof a therapeutically effective amount the compound as disclosed herein, or a pharmaceutical composition comprising the compound as disclosed herein.

More generally, the disclosed methods may be directed to inhibiting or inactivating an ornithine aminotransferase. Such a method can comprise providing a compound of the sort discussed above or described below, whether or not part of a pharmaceutical composition, and administering an effective amount of such a compound for contact with an ornithine aminotransferase. Such contact can be, as would be understood in the art, for experimental and/or research purposes or as may be designed to simulate one or more in vivo or physiological conditions. Such compounds can include but are not limited to those illustrated by the following examples, referenced figures, incorporated references and/or accompanying synthetic schemes. In certain such embodiments, such a compound and/or combination thereof can be present in an amount at least partially sufficient to inhibit OAT, cell proliferation and/or tumor growth.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 shows metabolic pathway of ornithine.

FIG. 2 depicts structures of selective hOAT inactivators and substituted analogues.

FIG. 3 shows plausible inactivation mechanisms for 17.

FIG. 4 shows proposed hydrogen bonding and salt bridge interactions for compound 17.

FIG. 5 shows concentration dependent results for compound 17 FIG. 6 shows Michaelis Menten Curve for compound 17.

FIG. 7 shows titration of hOAT with compound 17 The loss of enzyme activity is measured as a function of the ratio of inactivation to enzyme concentration.

FIG. 8 shows time-dependent dialysis of partially or fully inhibited hOAT by varying concentrations of 17.

FIG. 9 shows the hOAT and GABA-AT half-reactions.

FIG. 10 shows the theoretical pKa of γ and δ protons.

FIG. 11 shows the Polder map (Fo-Fc) at 4.0σ of the final adduct of hOAT inactivated by 17 (PDB ID: 8V9M). The top atom in close proximity to Tyr85 in the adduct is either a fluorine atom or a hydroxyl group.

FIG. 12 shows the final adduct displayed with the inactivator bound to PLP and Tyr85 and Phe177 closely contouring the complex. The surface feature distinguishes the van der Waals surface of each residue.

FIG. 13 shows the potential metabolites.

FIG. 14 shows the small molecule mass spectrometry analysis of metabolites.

FIG. 15 shows UV-vis spectra of hOAT with 17 and α-KG. Vertical arrows indicate the sequence of the observed spectra after initiating the experiment. The decay of the external aldimine intermediate (line 2), transient accumulation of the external aldimine intermediate (line 3), and subsequent turnover and decay of the external aldimine intermediate (line 4) are displayed FIG. 16A shows the kinetic studies of hOAT reacting with 17. A sample of hOAT (17 μM) was mixed with 17 (0-11 mM) using a stopped-flow spectrophotometer, and the reaction was observed between 300 and 800 nm for 150 sec using CCD detection. Several representative spectra from the data set are shown here.

FIG. 16B depicts the change in absorbance at the two peak maxima, 325 and 420 nm, with respect to time. These data were fit to a single step irreversible model using SVD.

FIG. 16C depicts the dependence of observed rate constants on the concentration of [5]. Fitting to a rectangular hyperbolic function (k_{obs}=kₗᵢₘᵢₜ[5]/K_{[5]}+[5] returned an observed limiting rate constant of 0.20±0.01 s⁻¹ and a dissociation constant for reversible formation of the external aldimine of 2.9±0.4 mM.

FIG. 17A shows the molecular docking binding pose of intermediate 23 in the molecular docking studies for intermediates in the active site of hOAT.

FIG. 17B shows the molecular docking binding pose of the external aldimine of 17a in the molecular docking studies for intermediates in the active site of hOAT.

FIG. 17C shows the molecular docking binding pose of the ketimine of 17a in the molecular docking studies for intermediates in the active site of OAT.

FIG. 17D shows the molecular docking binding pose of intermediate M1 in the molecular docking studies for intermediates in the active site of hOAT.

FIG. 18A shows the molecular docking binding pose of the external aldimine of 17 in the molecular docking studies for intermediates in the active site of pGABA-AT.

FIG. 18B shows the molecular docking binding pose of the external aldimine of 17a in the molecular docking studies for intermediates in the active site of pGABA-AT.

FIG. 18C shows the molecular docking binding pose of the ketimine of 17 in the molecular docking studies for intermediates in the active site of pGABA-AT.

FIG. 18D shows the molecular docking binding pose of the ketimine of 17a in the molecular docking studies for intermediates in the active site of pGABA-AT.

FIG. 19 shows the structure of hOAT homodimer inactivated by 17. The biological assembly is reconstructed by adding a crystallographically symmetric monomer (right side) to an observed monomer (left side) in an asymmetric unit using program Coot. The active site locations are indicated by the final adduct shown in space filling form.

FIGS. 20A and 20B show the comparison of hOAT-5 inactivated complex (FIG. 20B) and hOAT-AVA gem-diamine intermediate (FIG. 20A). Both structures display similarities in stabilization of carboxylate group of 17 and AVA with Tyr55, Arg180, and an ordered water.

FIG. 21 compares the mass spectrometry of native OAT with hOAT after inhibition by 17.

FIG. 22A shows the ¹⁹F NMR spectroscopy of hOAT inactivated by 17 under intact condition.

FIG. 22B shows the ¹⁹F NMR spectroscopy of the native hOAT.

FIG. 22C shows the ¹⁹F NMR spectroscopy of hOAT inactivated by 17 under denaturing condition.

FIG. 23 shows the mass spectrometry of the released PLP-17 complex.

FIG. 24 depicts the turnover number/partition ratio study of 17.

FIG. 25 depicts the ligand moiety of the final adduct is accessible to bulk solvent. The final adduct of hOAT inactivated by 17 is shown as ball-and-stick in the active site cavity.

### DETAILED DESCRIPTION OF THE INVENTION

The disclosed subject matter may be further described using definitions and terminology as follows. The definitions and terminology used herein are for the purpose of describing particular embodiments only, and are not intended to be limiting.

### Chemical Entities

New chemical entities and uses for chemical entities are disclosed herein. The chemical entities may be described using terminology known in the art and further discussed below.

As used herein, the term “halo” refers to a halogen substitution (e.g., —F, —Cl, —Br, or —I).

The term “ammonium” refers to the substituent

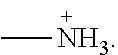

The term “carboxylate” as used herein refers to the substituent —C(O)O⁻.

The term “protic acid” as used herein refers to an acid that is able to release one or more protons and form hydronium ions in an aqueous solution. Examples of protic acid include, but are not limited to, monoprotic acids including hydrochloric acid (HCl), acetic acid (AcOH), nitric acid (HNO₃), benzoic acid (C₆H₅CO₂H), etc., diprotic acids including sulfuric acid (H₂SO₄), carbonic acid (H₂CO₃), hydrogen sulfide (H₂S), etc., triprotic acids including H₃PO₄. The term “conjugate base” refers to the anion that is formed after an acid loses one or more protons. One example of a conjugate base of a protic acid is chloride (Cl⁻).

### Compounds

The disclosed compounds may be directed to a compound of the following formula or a dissociated form, a non-protonated form, a zwitterion form, or a salt thereof:

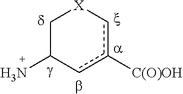

wherein a double bond is present between the α and ζ carbons, or between the α and β carbons, or not present between the α and ζ carbons and between the α and β carbons, X is CR¹R² or C═CR¹R², and R¹ and R² are independently hydrogen or halo (e.g., a halo such as F, Cl, Br, and I), providing at least one of R¹ and R² is halo.

In some embodiments, the double bond is between the α and ζ carbons. In such embodiments, the compound has a formula:

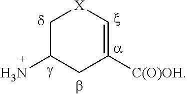

In some embodiments, the double bond is between the α and β carbons. In such embodiments, the compound has a formula:

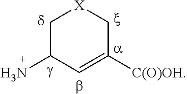

In some embodiments, a double bond is not present between the α and ζ carbons and between the α and β carbons. In such embodiments, the compound has a formula:

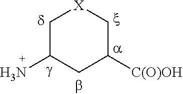

In some embodiments, X is CR¹R². In such embodiments, the compound has a formula:

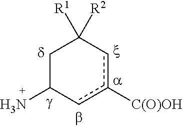

In some embodiments, X is C═CR¹R². In such embodiments, the compound has a formula:

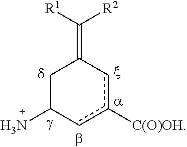

In some embodiments, X is CR¹R² and the double bond is between the α and ζ carbons. In such embodiments, the compound has a formula:

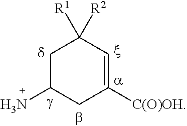

In some embodiments, X is CR¹R² and the double bond is between the α and β carbons. In such embodiments, the compound has a formula:

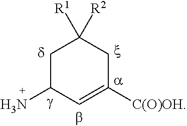

In some embodiments, at least one of R and R² is F. In some embodiments, R¹ and R² are F.

In some embodiments, the compound is a salt comprising a substituent that is an ammonium substituent or a carboxylate substituent. In some embodiments, the ammonium substituent has a counter ion that is a conjugate base of a protic acid (e.g. Cl⁻). In some embodiments, the carboxylate substituent has a counter ion that is an alkaline (such as Na⁺ and K⁺), alkaline-earth (such as Mg²⁺ and Ca²⁺) or ammonium cation.

In some embodiments, the compound is in zwitterion form comprising an ammonium moiety and a carboxylate moiety.

In some embodiments, the compound has a formula selected from the group consisting of

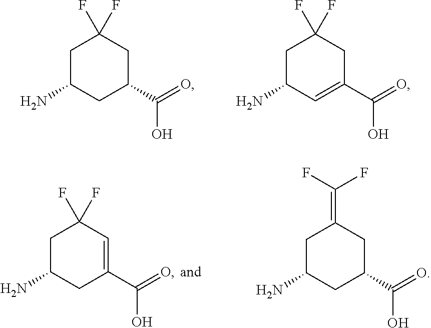

In some embodiments, the compound is

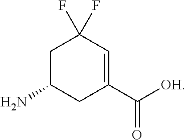 [SMILES: N[C@H]1CC(C(=O)O)=CC(F)(F)C1]

In some embodiments, the compound is

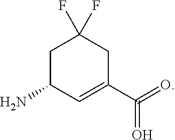

The compounds of the disclosure may be isomeric. In some embodiments, the disclosed compounds may be isomerically pure, wherein the compounds represent greater than about 99% of all compounds within an isomeric mixture of compounds. Also contemplated herein are compositions comprising, consisting essentially of, or consisting of an isomerically pure compound and/or compositions that are isomerically enriched, which compositions may comprise, consist essential of or consist of at least about 50%, 60%, 70%, 80%, 90%, 95%, 96%, 97%, 98%, 99%, or 100% of a single isomer of a given compound.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” or “+” or “−” depending on the configuration of substituents around the chiral or stereogenic carbon atom and or the optical rotation observed. The disclosed compounds encompasses various stereo isomers and mixtures thereof. Stereoisomers include enantiomers and diastereomers. Mixtures of enantiomers or diastereomers may be designated (+)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise. Also contemplated herein are compositions comprising, consisting essentially of, or consisting of an enantiopure compound and/or compositions that are enantiomer enriched, which compositions may comprise, consist essential of, or consist of at least about 50%, 60%, 70%, 80%, 90%, 95%, 96%, 97%, 98%, 99%, or 100% of a single enantiomer of a given compound (e.g., at least about 95% of an R enantiomer of a given compound).

### Pharmaceutical Compositions

Various non-limiting embodiments of the disclosed compounds and methods of use can be considered with an understanding of a catalytic mechanism of OAT and mechanism of inactivation of OAT. In some embodiments, the disclosed subject matter relates to one or more OAT inhibitors, as set forth above, formulated into compositions together with one or more physiologically tolerable or acceptable diluents, carriers, adjuvants or vehicles that are collectively referred to herein as carriers. Compositions suitable for such contact or administration can comprise physiologically acceptable aqueous or nonaqueous solutions, dispersions, suspensions or emulsions, whether or not sterile. The resulting compositions can be, in conjunction with the various methods described herein, for administration or contact with an ornithine aminotransferase. Whether or not in conjunction with a pharmaceutical composition, “contacting” means that an ornithine aminotransferase and one or more inhibitor compounds are brought together for purpose of binding and/or complexing such an inhibitor compound to the enzyme. Amounts of a compound effective to inhibit an ornithine aminotransferase may be determined empirically, and making such determinations is within the skill in the art. Inhibition or otherwise affecting an ornithine aminotransferase activity includes reduction, mitigation and/or modulation, as well as elimination of OAT activity, glutamate production, glutamine synthesis, cell proliferation and/or tumor growth.

In some embodiments, a pharmaceutical composition comprising the compound of as disclosed herein and a pharmaceutically suitable carrier, diluent, or excipient is provided.

The pharmaceutical composition may include the compound in a range of about 0.1 to 2000 mg. In some embodiments, the pharmaceutical composition may include the compound in a range of from about 0.5 to 500 mg. In some embodiments, the pharmaceutical composition may include the compound in a range of from about 1 to 100 mg. The pharmaceutical composition may be administered to provide the compound at a daily dose of about 0.1 to about 1000 mg/kg body weight. In some embodiments, the pharmaceutical composition may be administered to provide the compound at a daily dose of about 0.5 to about 500 mg/kg body weight. In some embodiments, the pharmaceutical composition may be administered to provide the compound at a daily dose of about 50 to about 100 mg/kg body weight. In some embodiments, after the pharmaceutical composition is administered to a subject (e.g., after about 1, 2, 3, 4, 5, or 6 hours post-administration), the concentration of the compound at the site of action may be within a concentration range bounded by end-points selected from 0.001 μM, 0.005 μM, 0.01 μM, 0.5 μM, 0.1 μM, 1.0 μM, 10 μM, and 100 μM (e.g., 0.1 μM-1.0 μM).

The compounds utilized in the methods disclosed herein may be formulated as a pharmaceutical composition that includes a carrier. For example, the carrier may be selected from the group consisting of proteins, carbohydrates, sugar, talc, magnesium stearate, cellulose, calcium carbonate, and starch-gelatin paste.

The compounds utilized in the methods disclosed herein may be formulated as a pharmaceutical composition that includes one or more binding agents, filling agents, lubricating agents, suspending agents, sweeteners, flavoring agents, preservatives, buffers, wetting agents, disintegrants, and effervescent agents. Filling agents may include lactose monohydrate, lactose anhydrous, and various starches, examples of binding agents are various celluloses and crosslinked polyvinylpyrrolidone, microcrystalline cellulose, such as Avicel® PH101 and Avicel® PH102, microcrystalline cellulose, and silicified microcrystalline cellulose (ProSolv SMCC™). Suitable lubricants, including agents that act on the flowability of the powder to be compressed, may include colloidal silicon dioxide, such as Aerosil®200, talc, stearic acid, magnesium stearate, calcium stearate, and silica gel. Examples of sweeteners may include any natural or artificial sweetener, such as sucrose, xylitol, sodium saccharin, cyclamate, aspartame, and acsulfame Examples of flavoring agents are Magnasweet® (trademark of MAFCO), bubble gum flavor, and fruit flavors, and the like. Examples of preservatives may include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Suitable diluents may include pharmaceutically acceptable inert fillers, such as microcrystalline cellulose, lactose, dibasic calcium phosphate, saccharides, and mixtures of any of the foregoing. Examples of diluents include microcrystalline cellulose, such as Avicelc® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

The compounds utilized in the methods disclosed herein may be administered in conventional dosage forms prepared by combining the active ingredient with standard pharmaceutical carriers or diluents according to conventional procedures well known in the art. These procedures may involve mixing, granulating and compressing or dissolving the ingredients as appropriate to the desired preparation.

Pharmaceutical compositions comprising the compounds may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips, or oil-in-water liquid emulsions or water-in-oil liquid emulsions.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis.

Pharmaceutical compositions adapted for topical administration may be formulated as ointments, creams, suspensions, lotions, powders, solutions, pastes, gels, impregnated dressings, sprays, aerosols or oils and may contain appropriate conventional additives such as preservatives, solvents to assist drug penetration and emollients in ointments and creams.

For applications to the eye or other external tissues, for example the mouth and skin, the pharmaceutical compositions are in some embodiments applied as a topical ointment or cream.

When formulated in an ointment, the compound may be employed with either a paraffinic or a water-miscible ointment base. Alternatively, the compound may be formulated in a cream with an oil-in-water cream base or a water-in-oil base. Pharmaceutical compositions adapted for topical administration to the eye include eye drops where the active ingredient is dissolved or suspended in a suitable carrier, especially an aqueous solvent.

Pharmaceutical compositions adapted for topical administration in the mouth include lozenges, pastilles and mouth washes.

Pharmaceutical compositions adapted for rectal administration may be presented as suppositories or enemas.

Pharmaceutical compositions adapted for nasal administration where the carrier is a solid include a coarse powder having a particle size (e.g., in the range 20 to 500 microns) which is administered in the manner in which snuff is taken (i.e., by rapid inhalation through the nasal passage from a container of the powder held close up to the nose). Suitable formulations where the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurized aerosols, nebulizers or insufflators.

Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solutions which may contain anti-oxidants, buffers, bacteriostats and solutes which render the formulation isotonic with the blood of the intended recipient, and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tableting lubricants, for example magnesium stearate, talc, polyethylene glycol or silica; disintegrants, for example potato starch, or acceptable wetting agents such as sodium lauryl sulphate. The tablets may be coated according to methods well known in normal pharmaceutical practice. Oral liquid preparations may be in the form of for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavoring or coloring agents.

Optionally, the disclosed compounds or pharmaceutical compositions comprising the disclosed compounds may be administered with additional therapeutic agents, optionally in combination, in order to treat cell proliferative diseases and disorders. In some embodiments of the disclosed methods, one or more additional therapeutic agents are administered with the disclosed compounds or with pharmaceutical compositions comprising the disclosed compounds, where the additional therapeutic agent is administered prior to, concurrently with, or after administering the disclosed compounds or the pharmaceutical compositions comprising the disclosed compounds. In some embodiments, the disclosed pharmaceutical composition is formulated to comprise the disclosed compounds and further to comprise one or more additional therapeutic agents, for example, one or more additional therapeutic agents for treating cell proliferative diseases and disorders.

It is understood by those skilled in the art that dosage amount will vary with the activity of a particular inhibitor compound, disease state, route of administration, duration of treatment, and like factors well-known in the medical and pharmaceutical arts. In general, a suitable dose will be an amount which is the lowest dose effective to produce a therapeutic or prophylactic effect. If desired, an effective dose of such a compound, pharmaceutically acceptable salt thereof, or related composition may be administered in two or more sub-doses, administered separately over an appropriate period of time.

Methods of preparing pharmaceutical formulations or compositions include the step of bringing an inhibitor compound into association with a carrier and, optionally, one or more additional adjuvants or ingredients. For example, standard pharmaceutical formulation techniques can be employed, such as those described in Remington's Pharmaceutical Sciences, Mack Publishing Company, Easton, PA.

### Methods

Generally, with respect to various embodiments, the disclosed subject matter can be directed to method(s) for the treatment of a pathologic proliferative disorder. As used herein, the term “disorder” refers to a condition in which there is a disturbance of normal functioning. A “disease” is any abnormal condition of the body or mind that causes discomfort, dysfunction, or distress to the person affected or those in contact with the person. Sometimes the term is used broadly to include injuries, disabilities, syndromes, symptoms, deviant behaviors, and atypical variations of structure and function, while in other contexts these may be considered distinguishable categories. It should be noted that the terms “disease”, “disorder”, “condition” and “illness”, are equally used herein.

In some embodiments, the compounds disclosed herein may function as mechanism based enzyme inactivators (MBEI) of OAT. An MBEI is an inert compound that is converted to an active intermediate by an enzyme's normal catalytic machinery. This active intermediate then can form a covalent bond with the enzyme itself or can bind tightly. (See, e.g., Silverman, R. B. “[10] Mechanism-Based Enzyme Inactivators.” Methods in enzymology 1995, 249, 240-283; the content of which is incorporate herein by reference in its entirety). Preferably, the disclosed compounds inactivate OAT with a rate of inactivation (k_{inact}) of greater than about 0.01 min⁻¹, 0.05 min⁻¹, 0.1 min⁻¹, 0.15 min⁻¹, 0.5 min⁻¹, 1.0 min⁻¹, 5.0 min⁻¹, 10 min⁻¹, or higher. Preferably, the disclosed compounds inactivate OAT and have an inhibition constant (K_{I}) of less than about 5 mM, 1 mM, 0.5 mM, 0.1 mM, 0.05 mM, 0.01 mM, 0.005 mM, 0.001 mM, or lower.

According to certain embodiments, the disclosed methods can be specifically applicable for the treatment of malignant proliferative disorders, including malignant proliferative disorders that express ornithine aminotransferase (OAT). As used herein, “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. Accordingly, the compounds, compositions, and methods disclosed herein may be used in the treatment of non-solid and solid tumors.

Malignancy, as contemplated herein, may be selected from the group consisting of melanomas, carcinomas, leukemias, lymphomas and sarcomas, which express OAT. Malignancies that can be treated by the methods disclosed herein, including malignancies that express OAT can comprise but are not limited to hematological malignancies (including leukemia, lymphoma and myeloproliferative disorders), hypoplastic and aplastic anemia (both virally induced and idiopathic), myelodysplastic syndromes, all types of paraneoplastic syndromes (both immune mediated and idiopathic) and solid tumors (including bladder, rectum, stomach, cervix, ovarian, renal, lung, liver, breast, colon, prostate, GI tract, pancreas and Karposi). More particularly, according to certain embodiments, the compounds and compositions used in conjunction can be used in methods for the treatment or inhibition of non-solid cancers, e.g. hematopoietic malignancies such as all types of leukemia, e.g. acute lymphocytic leukemia (ALL), acute myelogenous leukemia (AML), chronic lymphocytic leukemia (CLL), chronic myelogenous leukemia (CML), myelodysplastic syndrome (MDS), mast cell leukemia, hairy cell leukemia, Hodgkin's disease, non-Hodgkin's lymphomas, Burkitt's lymphoma and multiple myeloma, as well as for the treatment or inhibition of solid tumors such as tumors in lip and oral cavity, pharynx, larynx, paranasal sinuses, major salivary glands, thyroid gland, esophagus, stomach, small intestine, colon, colorectum, anal canal, liver, gallbladder, extraliepatic bile ducts, ampulla of Vater, exocrine pancreas, lung, pleural mesothelioma, bone, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma, retinoblastoma, carcinoma of the lacrimal gland, sarcoma of the orbit, brain, spinal cord, vascular system, hemangiosarcoma and Kaposi's sarcoma. The compounds and compositions disclosed herein may be administered in methods of treatment as known in the art. Accordingly, various such compounds and compositions can be administered in conjunction with such a method in any suitable way. For example, administration may comprise oral, intravenous, intraarterial, intramuscular, subcutaneous, intraperitoneal, parenteral, transdermal, intravaginal, intranasal, mucosal, sublingual, topical, rectal or subcutaneous administration, or any combination thereof.

According to some embodiments, the treated subject may be a mammalian subject. Although the methods disclosed herein are particularly intended for the treatment of proliferative disorders in humans, other mammals are included. By way of non-limiting examples, mammalian subjects include monkeys, equines, cattle, canines, felines, mice, rats and pigs.

The terms “treat, treating, treatment” as used herein and in the claims mean ameliorating one or more clinical indicia of disease activity in a subject having a pathologic disorder. “Treatment” refers to therapeutic treatment. Those in need of treatment are mammalian subjects suffering from any pathologic disorder. By “patient” or “subject in need” is meant any mammal for which administration of a compound or any pharmaceutical composition of the sort described herein is desired, in order to prevent, overcome, modulate or slow down such infliction. To provide a “preventive treatment” or “prophylactic treatment” is acting in a protective manner, to defend against or prevent something, especially a condition or disease.

As used herein, the term “a subject in need thereof” refers to a human or non-human subject that can be treated with any of the compounds or pharmaceutical compositions disclosed herein when the compounds or pharmaceutical compositions are utilized as therapeutic agents. A subject in need thereof may include a subject having a disease or disorder that is associated with a biological activity of ornithine aminotransferase (OAT). In some embodiments of the disclosed subject matter, a subject in need thereof may include a subject having a disease or disorder that is associated with increased expression of OAT (e.g., a subject having a cancer which overexpresses OAT).

A subject in need thereof may include a subject having a disease or disorder that is associated with ornithine aminotransferase (OAT), for example a disease or disorder that is associated with expression of OAT (e.g., increased expression of OAT) or a disease or disorder that is associated with a biological activity of OAT (e.g., increased activity of OAT for catalyzing synthesis of glutamate and/or glutamine). Diseases and disorders associated with expression of OAT and diseases or disorders that are associated with a biological activity of OAT are known and may include, but are not limited to cell proliferative diseases or disorders such as cancers.

In some embodiments, a subject in need thereof may include a subject having cancer. A subject in need thereof may include a subject having a cancer selected from, but not limited to, liver cancer (e.g., hepatocellular carcinoma (HCC)), and cancers such as multiple myeloma, leukemia, non-small cell lung cancer, colon cancer, cancer of the central nervous system, melanoma, ovarian cancer, renal cancer, prostate cancer, breast cancer, and pancreatic cancer.

More generally, the disclosed methods may be directed to affecting, modulate, reducing, inhibiting and/or preventing the initiation, progression and/or metastasis (e.g., from the liver elsewhere or to the liver from any other organ or tissue) of a malignant pathologic proliferative disorder associated with OAT activity. (See, e.g., Lucero O M, Dawson D W, Moon R T, et al. A re-evaluation of the “oncogenic” nature of Wnt/beta-catenin signaling in melanoma and other cancers. Curr Oncol Rep 2010, 12, 314-318; Liu Wei; Le Anne; Hancock Chad; Lane Andrew N; Dang Chi V: Fan Teresa W-M: Phang James M. Reprogramming of proline and glutamine metabolism contributes to the proliferative and metabolic responses regulated by oncogenic transcription factor c-MYC. Proc. Natl. Acad. Sci. USA 2012, 109(23), 8983-8988; and Tong, Xuemei; Zhao, Fangping; Thompson, Craig B. The molecular determinants of de novo nucleotide biosynthesis in cancer cells. Curr. Opin. Genet Devel. 2009, 19(1), 32-37.)

As used herein, the phrase “effective amount” shall mean that drug dosage that provides the specific pharmacological response for which the drug is administered in a significant number of patients in need of such treatment. An effective amount of a drug that is administered to a particular patient in a particular instance will not always be effective in treating the conditions/diseases described herein, even though such dosage is deemed to be a therapeutically effective amount by those of skill in the art.

As used herein, the term “modulate” means decreasing or inhibiting and/or increasing or augmenting. For example, modulating OAT activity may mean increasing or augmenting OAT activity, respectively, and/or decreasing or inhibiting OAT activity, respectively. The compounds disclosed herein may be administered to a subject in need thereof in order to modulate OAT activity, for example in order to inhibit OAT activity, where the subject has a disease or disorder associated with OAT activity. Ornithine aminotransferase (OAT) activity may be measured using methods disclosed herein and known in the art. (See, e.g., U.S. Published Application Nos. 2018/0098952, 20160128958, and 2012/0245380; and U.S. Pat. Nos. 8,686,041, and 8,211,865; the contents of which are incorporated herein by reference in their entireties).

Regardless of composition or formulation, those skilled in the art will recognize various avenues for medicament administration, together with corresponding factors and parameters to be considered in rendering such a medicament suitable for administration. Accordingly, with respect to one or more non-limiting embodiments, the disclosed compounds may be utilized as inhibitor compounds for the manufacture of a medicament for therapeutic use in the treatment or prevention of a disease or disorder associated with OAT activity, expression, or overexpression. Suitable diseases or disorders may include cell proliferative diseases or disorders, which may include but are not limited to hepatocellular carcinoma (HCC).

### MISCELLANEOUS

Unless otherwise specified or indicated by context, the terms “a”, “an”, and “the” mean “one or more.” For example, “a molecule” should be interpreted to mean “one or more molecules.” As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the claimed subject matter and does not pose a limitation on the scope of the claimed subject matter.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### ILLUSTRATIVE EMBODIMENTS

The following Embodiments are illustrative and should not be interpreted to limit the scope of the claimed subject matter.

Embodiment 1. A compound of the following formula or a dissociated form, a non-protonated form, a zwitterion form, or a salt thereof:

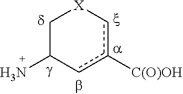

wherein
a double bond is present between the α and ζ carbons, or between the α and β carbons, or not present between the α and ζ carbons and between the α and β carbons;
X is CR¹R² or C═CR¹R²; and
R¹ and R² are independently hydrogen or halo, providing at least one of R¹ and R² is halo.

Embodiment 2. The compound of embodiment 1, wherein the double bond is between the α and ζ carbons.

Embodiment 3. The compound of embodiment 1, wherein the double bond is between the α and β carbons.

Embodiment 4. The compound of embodiment 1, wherein the double bond is not present between the α and ζ carbons and between the α and β carbons.

Embodiment 5. The compound of any one of embodiments 1-4, wherein X is CR¹R².

Embodiment 6. The compound of any one of embodiments 1-4, wherein X is C═CR¹R².

Embodiment 7. The compound of claim any one of embodiments 1-6, wherein at least one of R¹ and R² is F.

Embodiment 8. The compound of any one of embodiments 1-6, wherein R¹ and R² are F.

Embodiment 9. The compound of any one of embodiments 1-8, wherein the compound is a salt comprising a substituent that is an ammonium substituent or a carboxylate substituent.

Embodiment 10. The compound of embodiment 9, wherein the ammonium substituent has a counter ion that is a conjugate base of a protic acid.

Embodiment 11. The compound of any one of embodiments 1-8 in zwitterion form comprising an ammonium moiety and a carboxylate moiety.

Embodiment 12. The compound of embodiment 1 of a formula selected from the group consisting of:

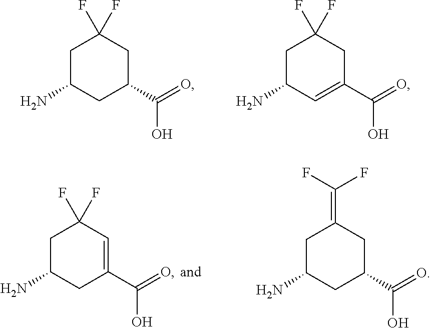

Embodiment 13. The compound of embodiment 1 of a formula that is:

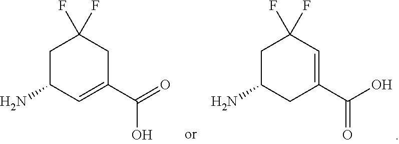 [SMILES: N[C@H]1C=C(C(=O)O)CC(F)(F)C1 ; N[C@H]1CC(C(=O)O)=CC(F)(F)C1]

Embodiment 14. A pharmaceutical composition comprising the compound of any one of embodiments 1-13 and a pharmaceutically suitable carrier, diluent, or excipient.

Embodiment 15. A method of modulating omithine aminotransferase (OAT) activity, the method comprising contacting the compound of any one of embodiments 1-13, or the pharmaceutical composition of embodiment 14, with a medium comprising an OAT, wherein the compound is present in an amount sufficient to modulate OAT activity.

Embodiment 16. The method of embodiment 15, wherein the contact is in vivo.

Embodiment 17. The method of embodiment 15 or 16, wherein the OAT is human ornithine aminotransferase (hOAT).

Embodiment 18. A method of reducing activity of an OAT expressed by a human cancer, the method comprising contacting the compound of any one of embodiments 1-13, or the pharmaceutical composition of embodiment 14, with the cancer expressing an OAT, wherein the compound is present in an amount that is effective to reduce OAT activity.

Embodiment 19. The method of embodiment 18, wherein the contact is in vivo.

Embodiment 20. The method of embodiment 18 or 19, wherein the contact is with a human subject in need thereof.

Embodiment 21. The method of any one of embodiments 18-20, wherein the OAT is human ornithine aminotransferase (hOAT).

Embodiment 22. A method for treating cancer in a subject in need thereof, the method comprising administering to the subject a therapeutically effective amount of the compound of any one of embodiments 1-13, or the pharmaceutical composition of embodiment 14.

Embodiment 23. The method of embodiment 22, wherein the cancer is characterized by expression or overexpression of human ornithine aminotransferase (hOAT).

Embodiment 24. The method of embodiment 22 or 23, wherein the cancer is hepatocellular carcinoma (HCC).

### Examples

The following Examples are illustrative and should not be interpreted to limit the scope of the claimed subject matter. The following non-limiting Examples and data illustrate various aspects and features relating to the disclosed compounds, compositions, and methods including the treatment of diseases and disorders associated with OAT activity, expression, or overexpression, and/or reduction of ornithine aminotransferase activity, such as cell proliferative diseases and disorders including, but not limited to hepatocellular carcinoma (HCC). While the utility of this invention is illustrated through the use of several compounds and compositions which can be used therewith, it will be understood by those skilled in the art that comparable results are obtainable with various other compound(s), as are commensurate with the scope of this invention.

Human ornithine aminotransferase (hOAT) is a pyridoxal 5′-phosphate (PLP)-dependent enzyme which is heavily involved in the synthesis of proline and glutamate. hOAT has been identified as a target for the treatment of hepatocellular carcinoma due to its impact on amino acid synthesis and the development of selective and potent pharmacological inhibitors is of significant interest. In this work, we designed, synthesized, and evaluated a series of substituted cyclohexene and cyclohexane compounds. Compound 17 is identified as time-dependent inhibitors of hOAT.

Hepatocellular carcinoma (HCC) accounts for around 75% of primary liver cancers and is in the top three causes of cancer death worldwide.¹ The most effective treatment for HCC is currently a liver transplant, with 5 year survival rates post-transplant being greater than 70%.² However, liver grafts are in limited supply leading to strict criteria for who can receive a transplant and wait times greater than 2 years in some parts of the US.³ Only 11.4% of HCC patients, and only 24.8% of patients with localized HCC, receive transplants withing 5 years of diagnosis, although in 2017~40% of all liver transplants in the US were for HCC.⁴ For patients who cannot receive a liver transplant the prognosis is far more grim, with the current standard of care being sorafenib, a multi-kinase inhibitor and radiotherapy. The overall 5-year survival rate for all patients with HCC is under 10%,⁵ and HCC recurrence is up to 80% within 5 years even after surgical resection.⁶ The prevalence and lethality of HCC necessitates the development of new effective treatment options.

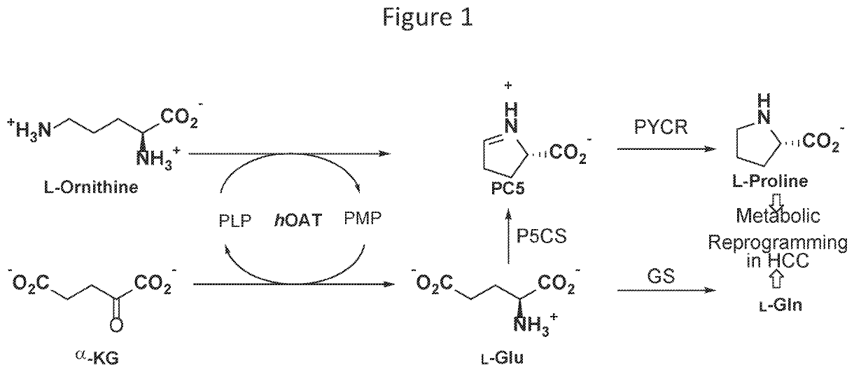

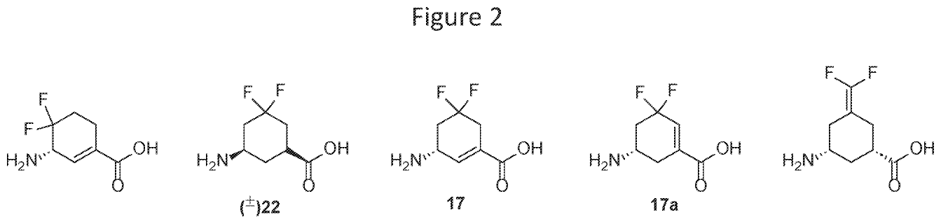

Human ornithine aminotransferase (hOAT) is a pyridoxal-5′-phosphate (PLP)-dependent enzyme⁷ that catalyzed the transfer of the delta-amino group from L-ornithine to alpha-ketoglutarate (alpha-KG) to provide glutamate-5-semialdehyde and glutamate respectively. hOAT is a mitochondrial enzyme that is involved in the production of amino acids glutamate and proline.⁸ Because hOAT is involved in amino acid synthesis, it also plays a role in the metabolism changes in and progression of HCC.⁹⁻¹⁰ hOAT operates through two coupled half-reactions to facilitate the transamination cycle (FIG. 1) In the first half-reaction, ornithine acts as the amino donor which is converted to glutamate-5-semialdehyde through the conversion PLP to pyridoxamine phosphate (PMP). The resulting glutamate-5-semialdehyde then spontaneously cyclizes to delta 1-pyrroline-5-carboxylate (P5C).¹¹ P5C can then be converted to proline by pyrroline-5-carboxylate reductase (PYCR).¹² In the second half reaction, PLP is regenerated from PMB by transferring the amino group to alpha-KG to yield L-glutamate (L-Glu), which can also be converted to P5C by pyrroline-5-carboxylate synthase (P5CS)¹¹ Proline metabolism appears to play a substantial role in metabolic reprogramming that sustains cancer proliferation and resistance.^{10, 13} Changes in the synthesis of PC5 and PYCR are associated with promoting cancer cell growth and resistance to apoptosis.¹⁴ The metabolic reprogramming in HCC, similar to other cancers, is characterized by activation of the proline/hydroxyproline pathway. This supports HIF1a-dependent tumor progression and plays a key role in HCC's resistance to sorafenib.¹⁵ Upregulation of other amino acids, such as glutamine, can also support unregulated cell growth, with upregulation of glutamine increasing de novo nucleotide synthesis and anabolic cell proliferation as well as the synthesis of glutathione (GSH) which acts as a major intracellular antioxidant.^{10, 16, 17} and the glutamate formed by hOAT can either be converted to proline or to glutamine by glutamine synthase (GS). hOAT and glutaminogenic enzyme are highly activated and often overexpressed in HCC due to aberrant, oncogenic Wnt/beta-catenin signaling.^{17,18} Selective inhibition of hOAT has been shown to effectively suppress the growth of HCC tumors in vivo.¹⁹ In 2019 the specific knockdown of hOAT was found to suppress the growth of nonsmall cell lung cancer in vitro and in vivo.²⁰ In 2022, it was found that methylation of hOAT may serve as an indicator for prognosis of postradiotherapy in oral squamous cell carcinoma,²¹ and that hOAT is overexpressed in gastric cancer patients.²² These results demonstrate the role hOAT plays in the metabolic reprograming of HCC as a key component of the proline and glutamine metabolic pathways as well as the therapeutic potential for selective inhibition of hOAT as a treatment for HCC and related cancers.

There are a variety of PLP dependent enzymes, including aminotransferases, decarboxylases, and transaminases. There are 14 known aminotransferase, and hOAT belongs to the same evolutionary subgroup within aminotransferases, the omega-aminotransferases,²³ as gamma-aminobutyric acid aminotransferase (GABA-AT) due to them both transferring an amino group that is distant from the alpha carbon instead of the more reactive alpha amino group.^{24, 25} The active sites of hOAT and GABA-AT share a high degree of homology, with the primary differences in the active sites being substitution of Tyr-85 and Tyr-55 in hOAT for an isoleucine and phenylalanine respectively in GABA-AT.²⁴ Tyr55 in hOAT is proposed to be a key binding residue for substrate ornithine; the α-amino group of ornithine is hydrogen bonded to Tyr55, leaving the S amino group to form a Schiff base with PLP^{2S}. Both enzymes share the key direct contacts between the enzyme and both the cofactor and substrate despite only sharing a 17% sequence homology.²⁴ Mechanism-based inactivators (MBIs) are unreactive compounds designed to engage with the enzyme and generate an active species in the catalytic pocket that subsequently will bind and inactivate the enzyme. This binding is functionally irreversible and can be covalent binding or a tight-binding adduct, providing irreversible inhibition on an enzyme-relevant timescale. Because MBIs are inert until they are engaged with the active site to generate a reactive species, the risk of undesirable off-target effects can be greatly reduced. More importantly, these inactivators can provide higher potency and selectivity than traditional inhibitors with lower dosages.

Herein, a series of potential hOAT inactivators designed to have hOAT specific mechanisms of inactivation are described. The activity of exemplary compounds 17a and 17 are explained below.

hOAT Inactivators

Due to the similar binding pockets of hOAT and GABA-AT, there are nearly identical distances between key anchor points for the ligands. One notable difference is that Tyr55 in hOAT, while occupying a similar steric space as Phe351 in GABA-AT, is far more hydrophilic and occupies less space overall due to the orientation of the aryl ring. This allows the pocket of hOAT to be larger and more flexible to accommodate ornithine.

The mechanism of aminotransferase MBIs may begin by engaging the enzyme in the same manner as the endogenous ligand. A Schiff base (aldimine) with the aldehyde of PLP previously bound to the Lys residue may be formed. A deprotonation event then converts the aldimine to an activated ketimine intermediate. This highly active intermediate can then undergo further steps to inactivate the enzyme. Ketimine is more stable in the active site of OAT compared to GABA-AT, which may allow more time for the inhibitor to further react with OAT prior to the unproductive hydrolysis pathway. Compound 17 was designed to undergo a second deprotonation event at the gamma-position after forming the initial internal ketimine complex. The gamma position may be activated by the adjacent fluorines and imine, which can enhance the acidity of the gamma-proton and promote the abstraction. The subsequent elimination of a fluoride ion would provide a quinoid intermediate that could have at least three fates: pathway a, aromatization or nucleophilic substitution by Lys292 (pathway b) or water (pathway c) with loss of the second fluoride ion. Pathway b results in covalent inactivation of hOAT, whereas pathways a and c lead to the formation of tightly bound inactivator-cofactor adducts that inactivate hOAT and are unlikely to be released from the pocket on a biologically relevant timescale.

MBIs generally act as a substrate during the initial binding, and the enzyme engages them to form reactive intermediates that can inactivate the enzyme. Some slow-tight binding inactivators can instead form a highly stable intermediate when engaged with the enzyme, preventing hydrolysis of the inactivator and essentially covalently binding to the protein or cofactor. While a covalent bond may form between the activated quinoid intermediate, the aromatization pathways would provide tightly bound inhibitor-cofactor complexes that are unlikely to hydrolyze from the pocket on a biologically relevant timescale.

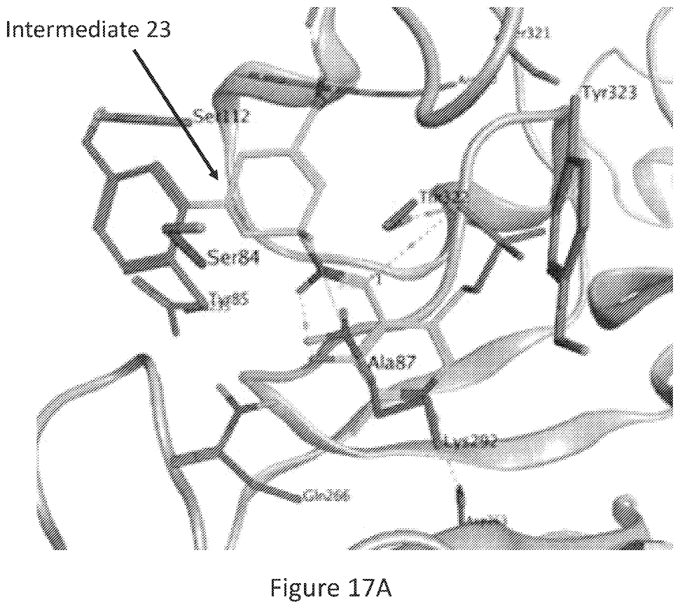

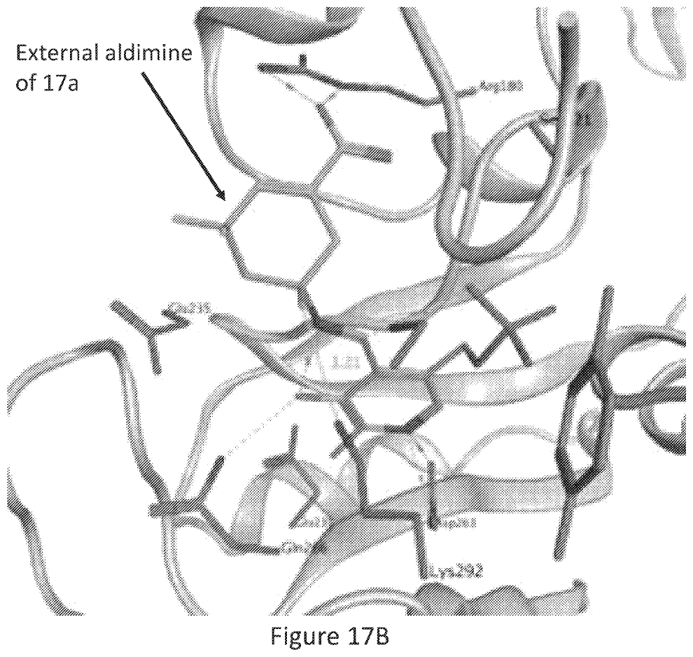

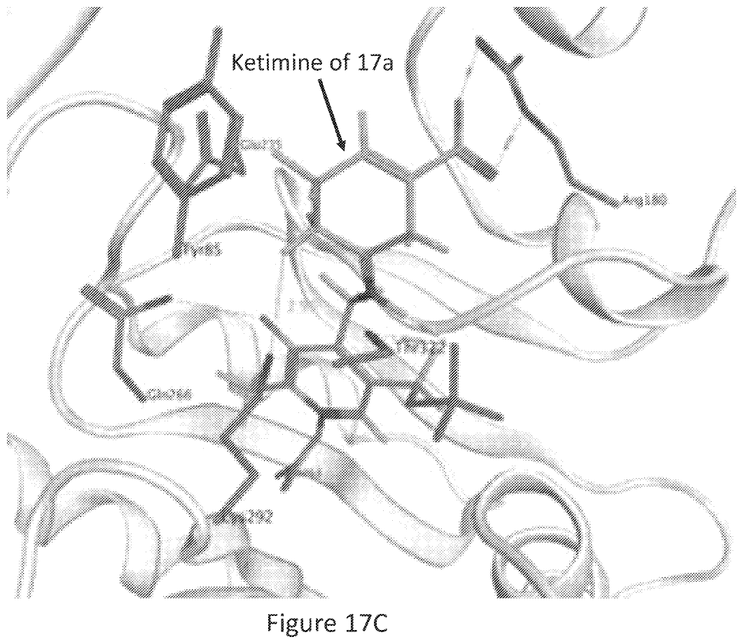

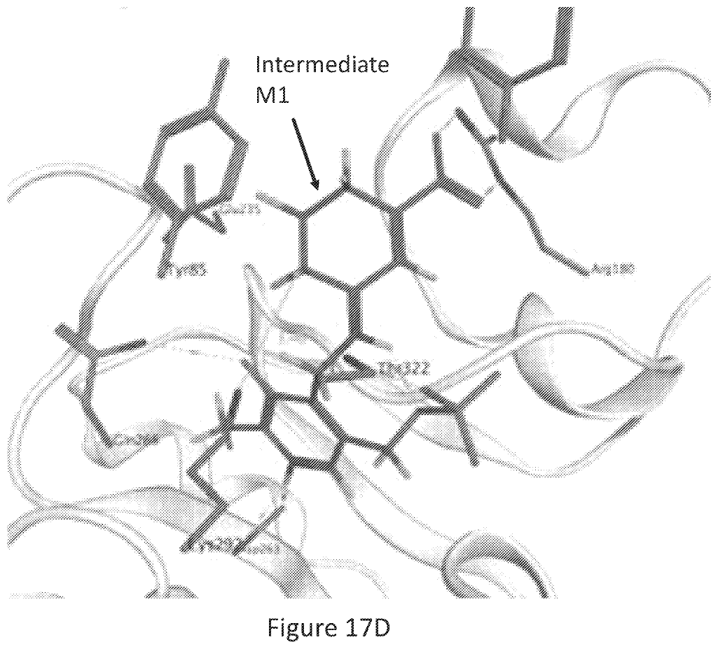

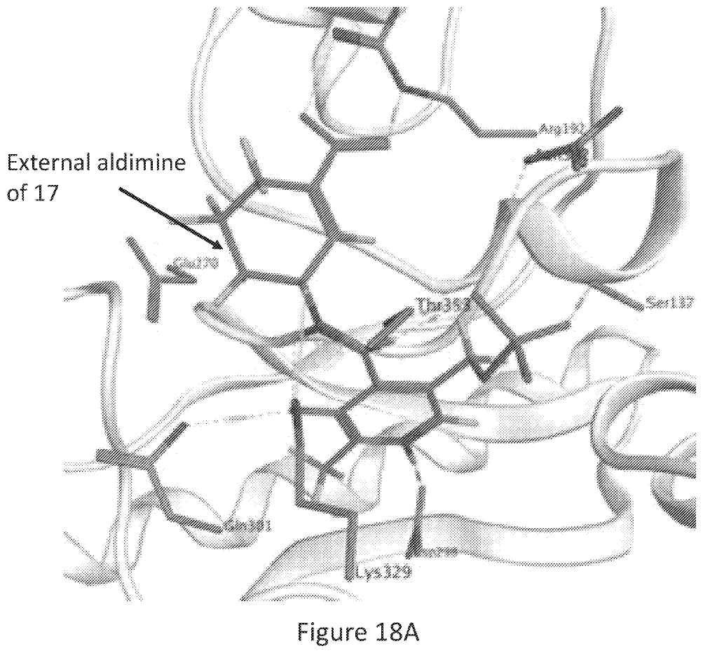

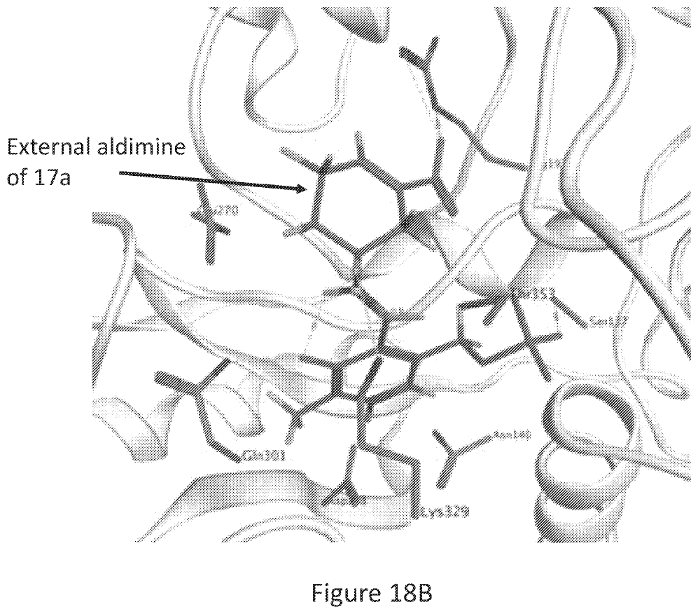

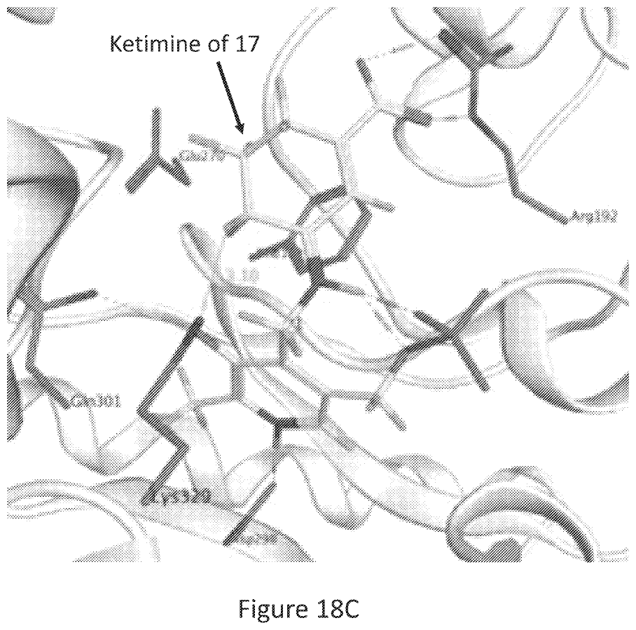

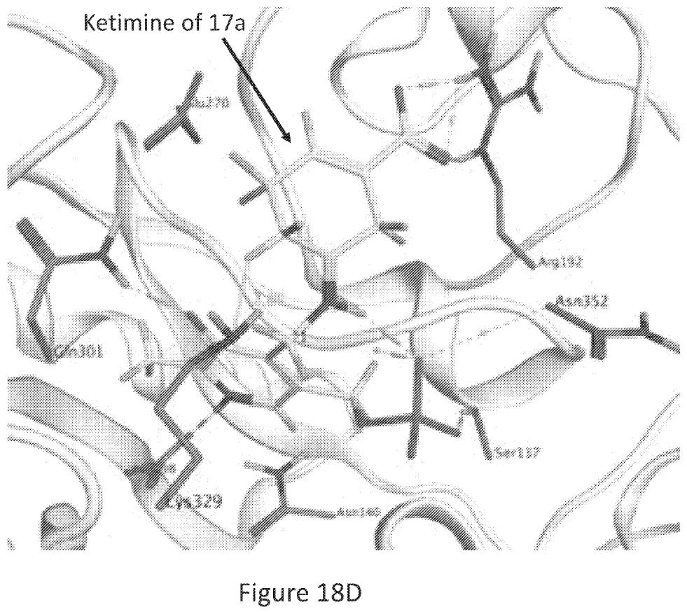

To evaluate the potential for the initial deprotonation of the external aldimine intermediate for both 17 and 17a, molecular docking studies were employed (FIGS. 17A-D and 18A-D). In hOAT, the external aldimine intermediates of both 17 and 17a showed similar binding poses, with the carboxylate of the inactivator moiety forming stable hydrogen bonds with Arg180. However only 17 formed an additional hydrogen bond with Tyr55. Both structures showed the γ-proton was in close proximity to Lys292 (3.0 Å for 17 and 3.2 Å for 17a) suggesting that the formation of the ketimine is reasonable. Next, the 6-deprotonation was evaluated. The modeling of the ketimine intermediate for both 17 and 17a predicted that Lys292 would be near the 6-hydrogen of the intermediate, suggesting both should be capable of undergoing the second elimination (3.5 Å for 17 and 3.0 Å for 17a).

### Syntheses of Substituted Analogs

The synthesis toward 1 begins with a previously reported iodolactonization of R-(+)-3-cyclohexenecarboxylic acid (4), followed by elimination of the iodine with DBU to form 5 (Scheme 1). Compound 5 was then allowed to react with n-bromoacetimide, yielding lactone 6, which was then debrominated with tributyltin hydride in the presence of catalytic AIBN at reflux to access lactone 7. Lactone 7 was opened with HCl in methanol at 40° C. overnight to facilitate alcohol 8 in quantitative yields. Alcohol 8 was able to be used without further purification but has limited solubility and is only able to be dissolved in alcohols and water. Oxidation of 8 with PCC in DCM provided ketone 9 as a light green solid in 68% yield with trace chromium contamination.

### Scheme 1. Synthesis of Inactivator

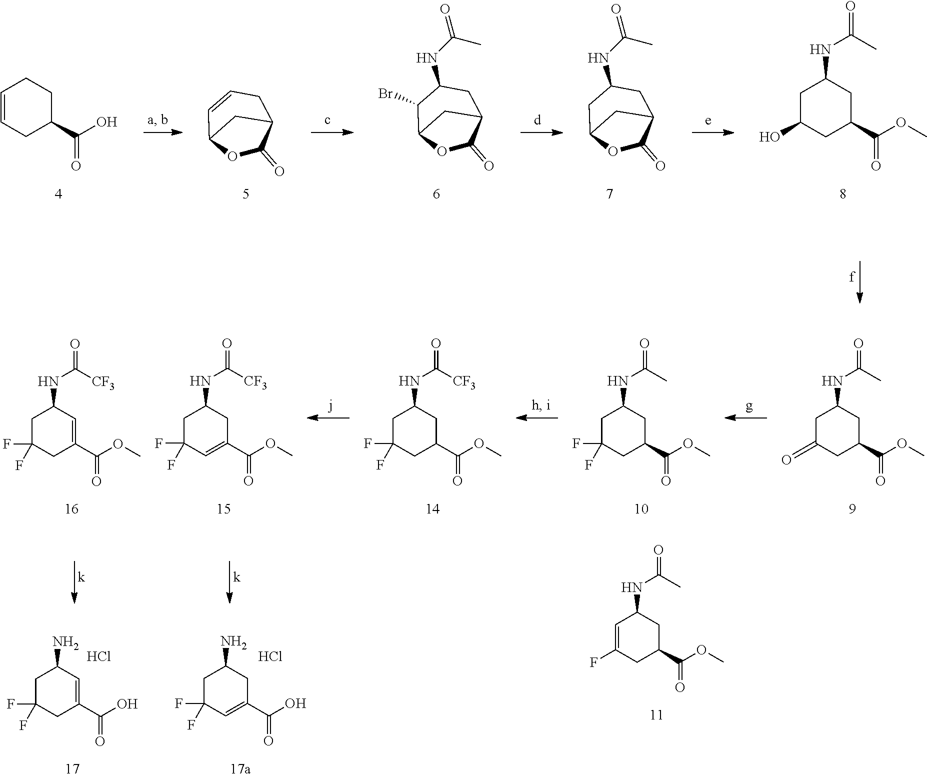

Conditions: (a) 12, overnight (b) DBU; (c) n-bromoacetamide; (d) Bu₃SnH, AIBN, 80° C., overnight; (e) HCl, MeOH, 40° C., overnight; (f) PCC, DCM, overnight; (g) DAST, DCM, 50° C., 3 h; (h) 3M HCl, MeOH, 75° C., 2 d; (i) TFAA, DCM, overnight; (j) KHMDS, PhSeBr, THF then mCPBA, DCM; (k) HCl (aq., 6 M), 90° C., overnight.

To achieve good yields, multiple extractions with ethyl acetate from an acidic aqueous phase was required. The yield is also highly dependent on 8 being well dispersed and suspended in the reaction solution due to the insolubility of 8 in DCM. Difluorination of 9 with DAST facilitated 10 in moderate yields along with the vinyl fluoride 11. These fluorinated products were challenging to separate with the acetyl protecting group and so were moved forward as a mixture. Deprotection of the acetyl was performed on a mixture of 10 and 11 with 4M HCl in methanol at reflux over 2 days. The resulting solution was directly concentrated and resuspended in DCM and allowed to stir with an excess of TFAA overnight. The two resulting trifluoroprotected analogs of 10 and 11 were then far more separable and 14 was cleanly isolated in 25% yield over the three steps. Improved yields would likely be obtained by improving the acetyl deprotection, which was sluggish and did not go to completion. However, this provided more than enough material to move forward with the synthesis. Selenation alpha to the ester was performed and the subsequent elimination with mCPBA furnished a mixture of alpha beta unsaturated esters 15 and 16 in 84% yield over two steps. The resulting regioisomers showed modest separation via reverse phase chromatography. The two regioisomers were characterized with 2D NMR and x-ray crystallography to confirm absolute stereochemistry. Previous work on similar scaffolds had shown that with a Boc protecting group the amine cyclizes onto the ester to form a lactam under these conditions. Due to the electron withdrawing effects of the trifluoroacetamide this was not observed in this system. When the Boc protected analog of 14 was used, formation of the lactam was observed, and the Boc protected analogs of 15 and 16 were not separable. Global deprotection of both 15 and 16 was done by refluxing in aqueous 4 M HCl to yield the desired HCl salts 17 and 17a in 34% and 87% yield respectively. Both 17a and 17 were then purified via reverse phase chromatography with a C18 aqueous column.

The racemic synthesis of the unsaturated analogues began with the diastereoselective hydrogenation of 3-amino-5-hydroxybenzoic acid hydrochloride 18 with rhodium on alumina to provide 19 as a single diastereomer. The acid was converted to the methyl ester by generating HCl with thionyl chloride in methanol. Lastly, the material was Boc-protected to afford racemic alcohol 19 in 37% yield over three steps. Oxidation of the alcohol group on 19 with PCC provided ketone 20 in 80% yield, which was then subjected to DAST to provide difluorinated intermediate 21 in 59% yield. Global deprotection of 21 provided saturated analogue (±)22 (Scheme 2).

### Scheme 2. Synthesis of the Saturated Analogue

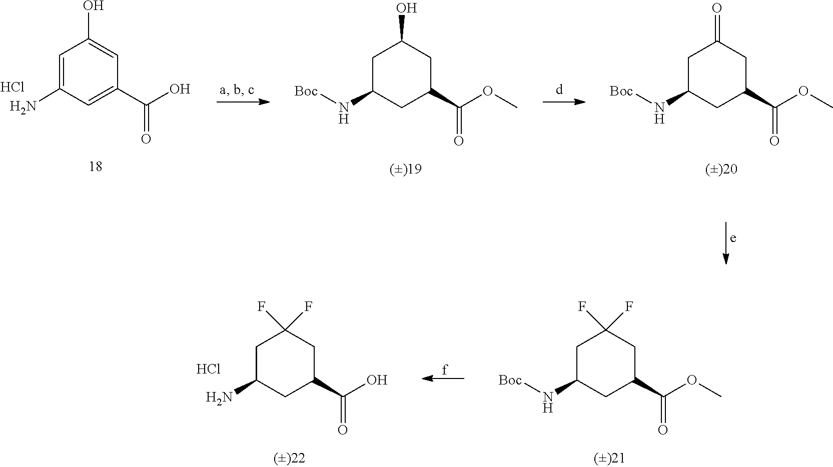

Conditions and Reagents: (a) Rh/Al₂O₃, H₂ (400 psi), 90° C., overnight; (b) SOCl₂, MeOH, r.t.; (c) Boc₂O, NaHCO₃, MeOH, r.t.; (d) PCC, DCM, overnight, r.t.; (e) DAST, 1,2-dichloroethane, 40° C., overnight; (f) 4 MHCl, AcOH, 80° C., overnight.

### Kinetic Studies of Analogs

Of the analogues studied, in vitro experiments demonstrated 17 had the greatest inhibitory effect against hOAT (Table 1) with an overall efficiency (k_{inact}/K_{I}) of 1.04 mM-min-. In addition, 17a displayed weaker inactivation effects against both hOAT and hGABA-AT with k_{inact}K_{I} values 10-fold less effective than that of 17 for hOAT. The double bond allows for inactivation, as the saturated analogue ((±) 22) displayed little or no inhibitory effect against hOAT or hGABA-AT.

TABLE 1
Kinetic constants for the inactivation of hOAT and hGABA-AT.

| | hOAT | | | hGABA-AT | | |
|---|---|---|---|---|---|---|
| | | k_{inact} | k_{inact}/K₁ | | k_{inact} | k_{inact}/K₁ |
| Compound | K₁ (mM) | (min⁻¹) | (mM⁻¹min⁻¹) | K₁ (mM) | (min⁻¹) | (mM⁻¹min⁻¹) |
| 17 | 0.27 ± 0.07 | 0.28 ± 0.1 | 1.04 | —^{a} | —^{a} | 0.17 |
| 17a | 1.2 ± 0.4 | 0.15 ± 0.02 | 0.11 | —^{a} | —^{a} | 0.12 |

| | | |
|---|---|---|
| (±)22 | No inhibition up to 10 mM | Weak reversible inhibition at 10 mM |

^{a}K₁ and K_{inact} unable to be determined for week inactivation

### Effects of the Endocyclic Alkene on the pKₐ of the δ-Proton

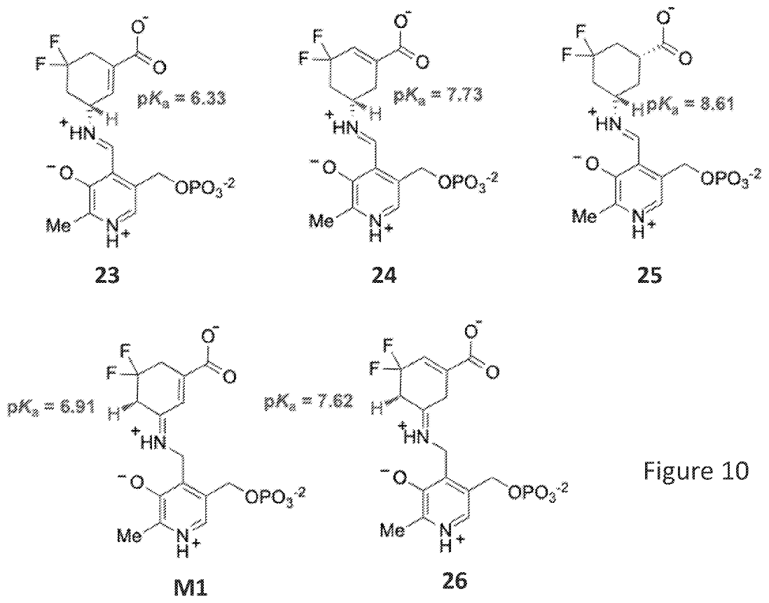

We hypothesize that the presence of the α,β-unsaturated alkene in 17 compared to 17a and (±)22 may have an influence on the acidity on both the γ and δ position. The proposed mechanisms of inactivation include two deprotonation events: first at the γ-position of the inactivator on the aldimine, and then the δ-position of the ketimine. It is common for the γ-deprotonation event, which initiates the inactivation mechanism, to be rate-limiting. However, in this mechanism another plausible rate-limiting step is the δ-deprotonation event. Regardless of which step is rate-limiting, the pKₐ of these two protons will play a role in the potency of these potential inactivators and their ability to engage with the target enzyme. To evaluate the effect of the unsaturation on the acidity of these protons, we applied theoretical pKₐ calculations for the corresponding intermediate states using the hybrid DFT/B3LYP method in Gaussian09 (FIG. 10). The proposed aldimine of the saturated analogue (25) had a higher theoretical pKₐ (8.6) for the γ-proton compared to the unsaturated analogues (23 and 24), indicative of the necessity for installation of the double bond to promote inactivation. When comparing 17 and 17a, a difference in the theoretical pKₐ was observed for both the γ-proton of the aldimine (6.33 vs 7.73) and δ-proton of the ketimine (6.91 vs 7.62). Based on the modeling studies shown in FIGS. 17A-D and 18A-D, the binding poses and distances between the catalytic Lys292 and the γ/δ protons for the deprotonation steps of both 17a and 17 are similar. This suggests that the observed difference in activity between 17a and 17 is affected, in part, by the relative acidity of these protons.

X-ray Crystallography of hOAT Inactivated by 17a and 17

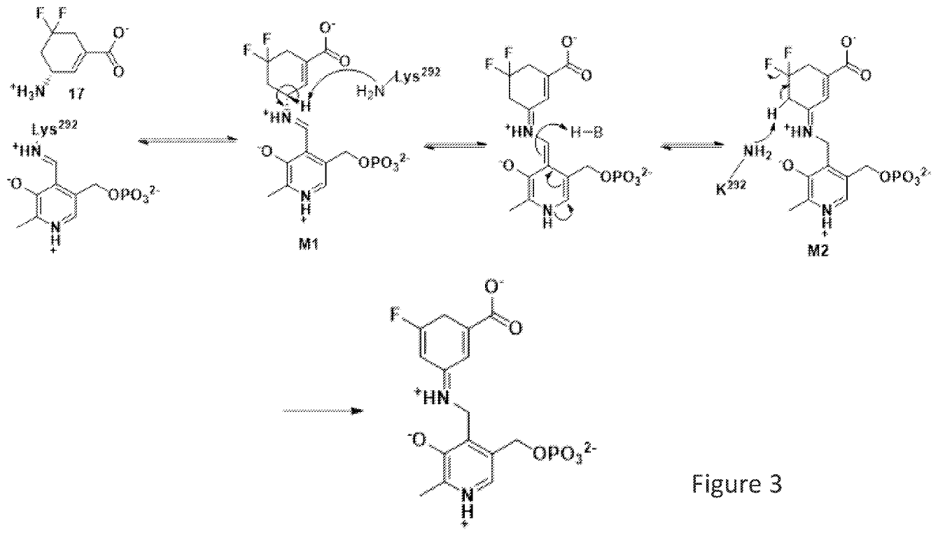

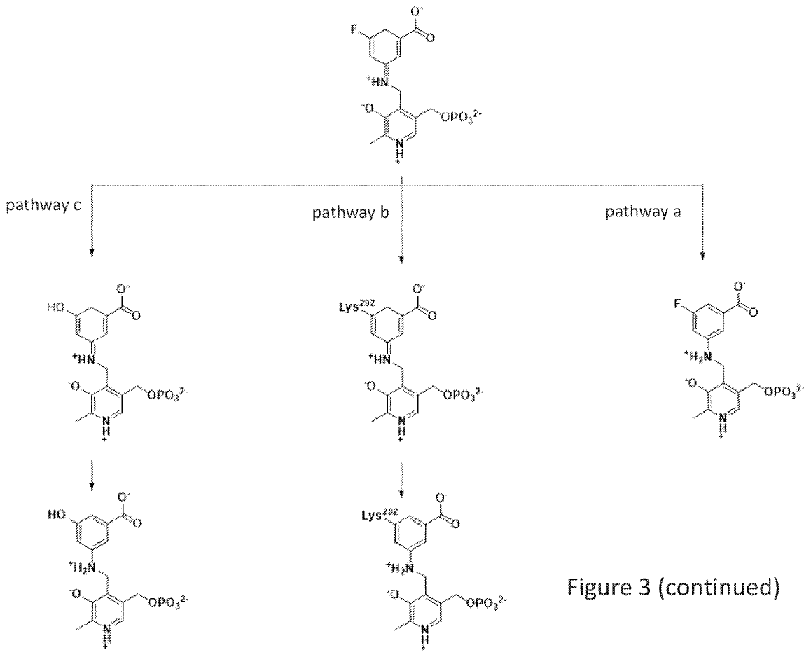

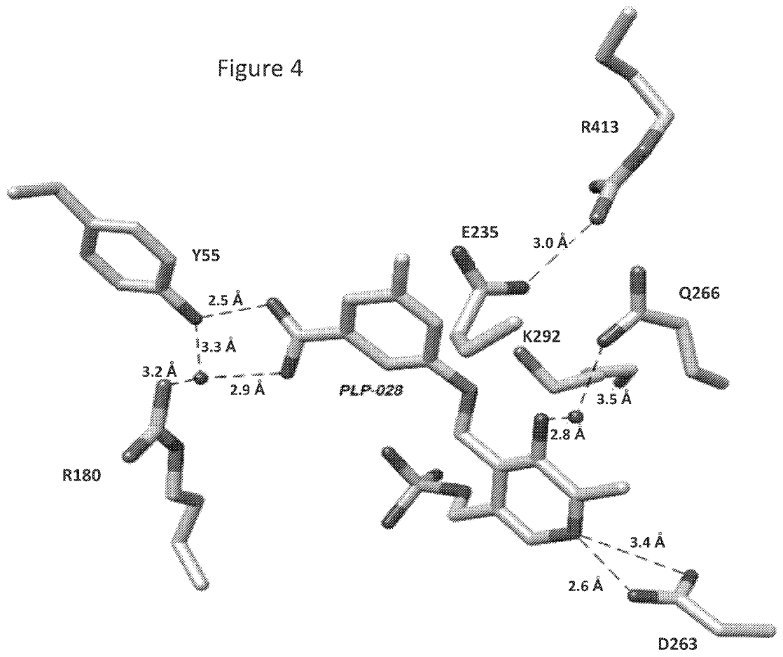

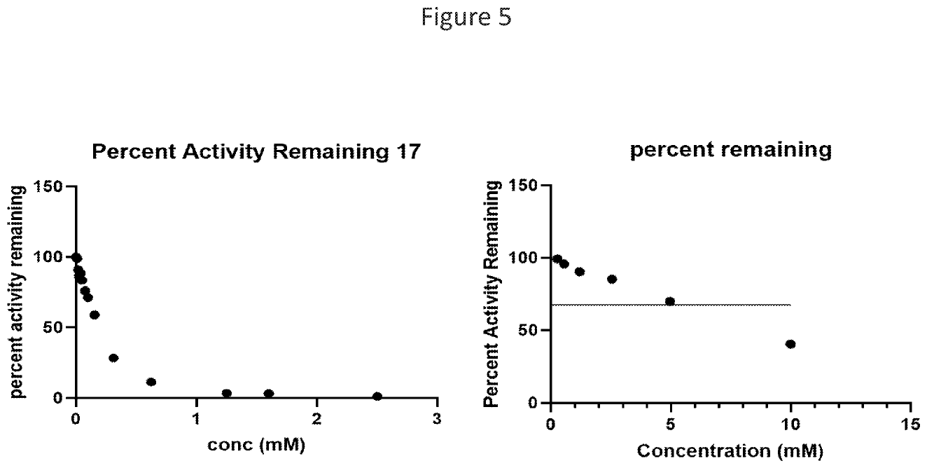

Protein crystallography of hOAT inactivated by 17 was conducted to investigate the adduct formed in the active site upon inactivation. As seen in FIG. 4, 17 forms a covalent bond to PLP but not Lys292. This does rule out pathway b as a possible mechanism of inactivation (FIG. 3). The electron density was not defined enough to determine if pathway a or pathway c is more likely. The E235-R⁴¹³ salt bridge is partially disrupted by the adduct, and it appears that glutamate forms an interesting van der Waals interaction with the potential fluorine atom on the inactivator.

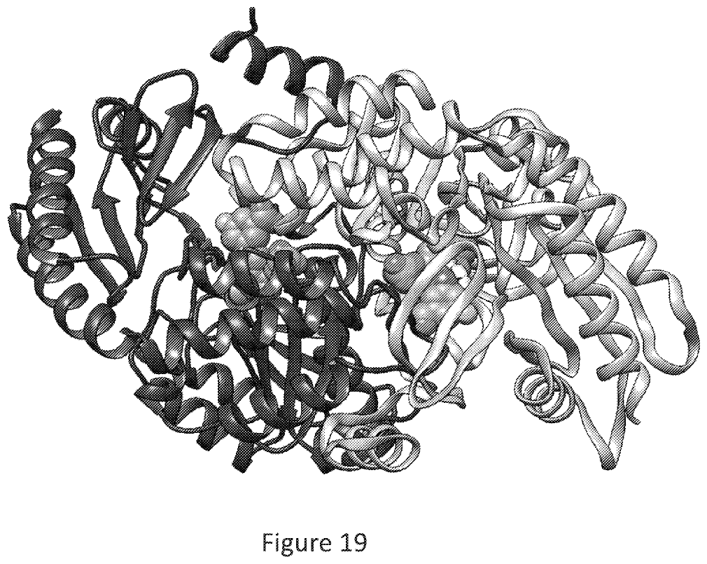

To examine the inactivated enzyme-17 complex directly, an X-ray crystal structure of hOAT inactivated by 17 was obtained. Following complete, irreversible inactivation of hOAT by 17, hOAT crystals were grown over 7 days which diffracted to 1.61 Å resolution. The structure of the hOAT-17 inactivated complex was solved utilizing a monomer from a previously published structure of hOAT (PDB code: 6V8C) as the starting model for molecular replacement. Following model building and refinement cycles, the refined molecular model contained three monomers in one asymmetric unit in the P3₃21 space group with R_{free}/R_{work} values of 19.30%/16.42% (Table 2). The homodimeric biological assembly of hOAT was reconstructed by the addition of a crystallographically symmetric monomer to give an observed monomer in the asymmetric unit (FIG. 19).

TABLE 2
Data Collection and Refinement Statistics

| | |
|---|---|
| PDB Code | 8V9M |

Data Processing

| Wavelength (Å) | 1.12718 | |
|---|---|---|
| Resolution range (Å) | 42.21-1.61 | |
| Space group | P 322₁ | |
| Unit cell dimensions (Å) | 115.334, 115.334, 186.292 | |
| Unit cell angles (°) | 90.0, 90.0, 120.0 | |
| Total reflections | 2516639 | |
| Unique reflections | 185048 | |
| Multiplicity | 13.5 | (13.3) |
| Completeness (%) | 99.96 | (99.99) |
| Mean I/Sigma(I) | 10.7 | (1.0) |
| Wilson B-factor | 23.46 | |
| ^{b}Rₚᵢₘ | 0.040 | (0.696) |
| ^{c}CC_{1/2} | 0.998 | (0.354) |

Refinement

| | | |
|---|---|---|
| ^{d}R_{work} | 0.1640 | (0.2840) |
| ^{e}R_{free} | 0.1927 | (0.3115) |

| | |
|---|---|
| Number of Non-Hydrogen Atoms | 10736 |
| Macromolecules | 9502 |
| Ligands | 102 |
| Solvent | 1132 |
| ^{f}RMS(bonds) (Å) | 0.015 |
| RMS(angles) (°) | 1.30 |
| Ramachandran favored (%) | 96.19 |
| Ramachandran allowed (%) | 3.65 |
| Ramachandran outliers (%) | 0.17 |
| Average B-factor (Å²) | 34.13 |
| Macromolecules (Å²) | 33.47 |
| Ligands (Å²) | 29.02 |
| Solvent (Å²) | 40.11 |

^{a}The values for the highest-resolution bin are in parentheses,
^{b}Precision-indicating merging R,
^{c}Pearson correlation coefficient of two “half” data sets,
^{d}R_{work} = Σ|F_{obs} − F_{calc}|/ΣF_{obs},
^{e}Five percent of the reflection data were selected at random as a test set, and only these data were used to calculate R_{free},
^{f}Root-mean square deviation.

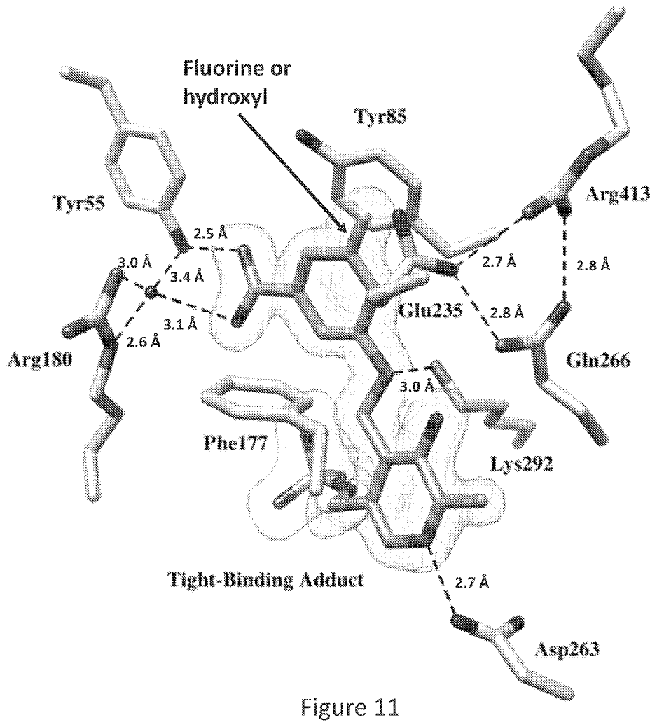

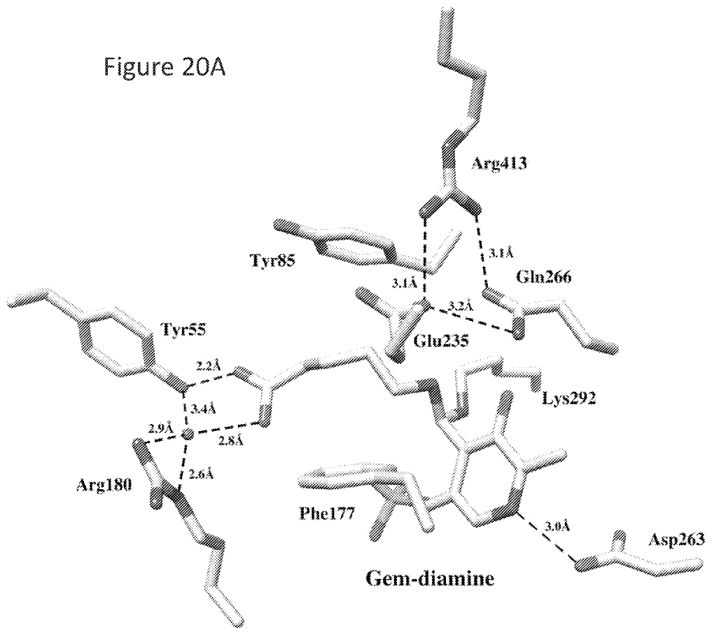

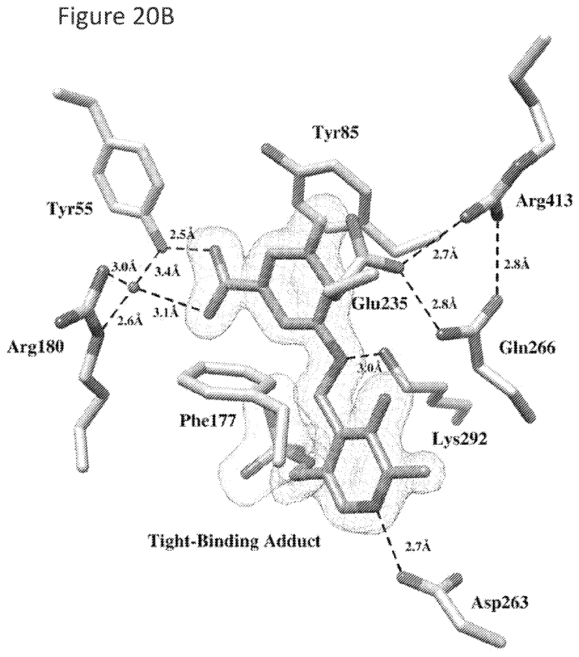

The observed electron density supports an inactivator covalently linked to PLP (FIG. 11). The observed electron density demonstrates that the binary complex is detached from Lys292 and other active site residues, excluding pathway b as a possible mechanism for inactivation. The oxygen atoms on the carboxylate group of the inactivator are stabilized through a hydrogen bonding network with Tyr55 and Arg180, whose native functions are to interact with the α-amino group and the α-carboxylate group of ornithine, respectively.²⁴ One carboxyl oxygen atom forms a strong hydrogen bond (2.5 Å) with Tyr55, and the other oxygen atom maintains an indirect hydrogen bonding interaction with both Tyr55 and Arg180 through an ordered water. Interestingly, a previously published crystal structure of 5-aminovaleric acid complexed with hOAT displayed a nearly identical stabilization of the carboxylate with Tyr55, Arg180, and a water molecule (FIGS. 20A and 20B).²⁸ 5-Aminovaleric acid is a slow-binding alternate substrate for hOAT and has been reported to bind in a different orientation compared with ornithine due to lack of an (α-amino group. Tyr55 plays a distinct role in the accommodation of the charged α-amino group of ornithine in the active site of hOAT when compared to the hydrophobic residue Phe351 in GABA-AT.²⁴ Therefore, the strong interaction of carboxylate in this cyclic GABA analogue with Tyr55 is highly important for the selectivity of 17 for hOAT over GABA-AT.

In two of the monomers within the same asymmetric unit, the Glu235-Arg413 salt bridge is partially disrupted but remains intact in one of the monomer copies. This salt bridge is proposed to function as a molecular switch between the first and second half-reactions, preventing nonproductive interaction between Arg413 and the carboxylate group of ornithine in the first half-reaction.²⁴ In the copies displaying the partially disrupted salt bridge, the side chain of Arg413 has rotated away from Glu235, most likely a result of the bulky cyclohexene ring scaffold of the inactivator.

### Dialysis

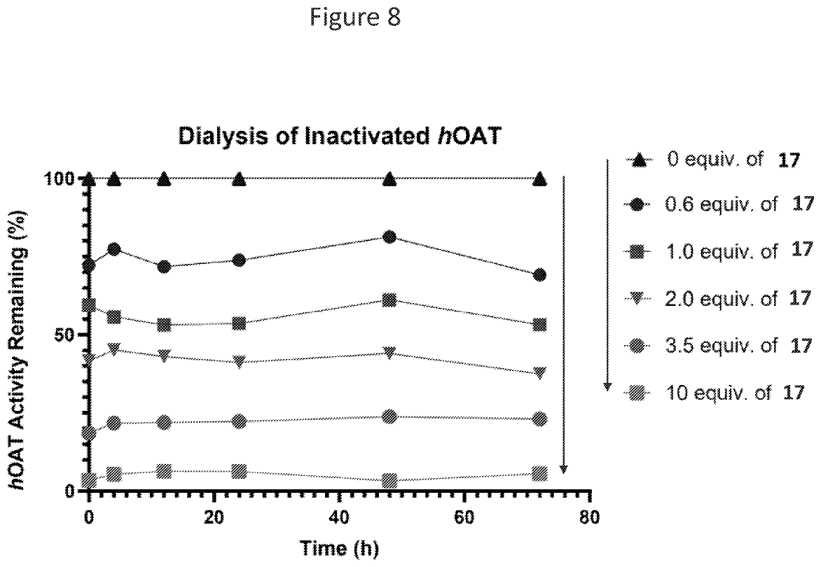

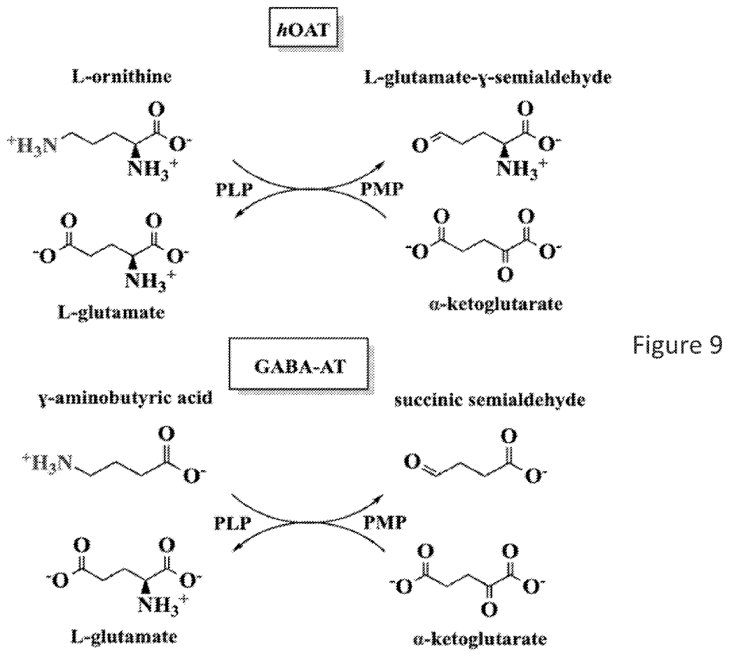

The crystal structure of 17 bound to hOAT (FIG. 11) does not differentiate pathway a from pathway c because of the inability to differentiate a fluorine atom from a hydroxyl group at that resolution. Mass spectrometry, however, might differentiate those possibilities. To confirm irreversible inactivation of hOAT by 17, a time-dependent reactivation experiment (dialysis) was performed. After hOAT activity was partially or fully decreased with 0.6-10 equivalents of 17, the samples were dialyzed. Aliquots at different time intervals were collected and assayed to detect restored enzyme activity. No enzymatic activity was recovered after 72 hours of dialysis (FIG. 8), indicating complete irreversible inhibition of hOAT by 17 on an enzymatically relevant timescale. It takes approximately 10 equiv of 17, however, to produce complete enzyme inactivation.

Mass Spectrometry of the Adduct Formed when hOAT is Inactivated by 17

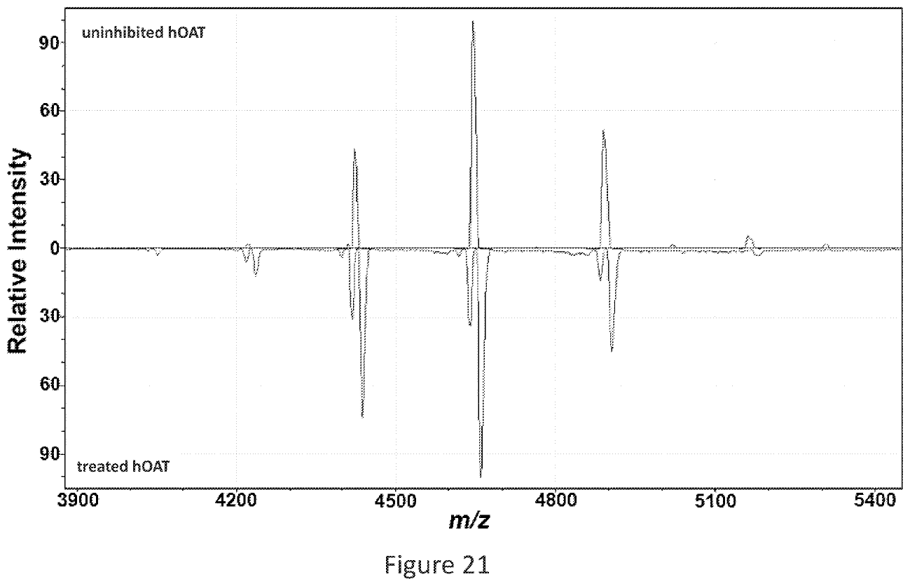

Intact protein MS was utilized to distinguish between a potential covalent (pathway h) or tight-binding (pathway a or c) inactivation mechanisms for hOAT by 17. Using native top-down protein mass spectrometry, the molecular mass of native hOAT was compared to the molecular mass of hOAT after complete inhibition by 17. A mass shift was observed that corresponded to the addition of the inhibitor, confirming that the inhibitor was bound to the protein/PLP complex (FIG. 21). Next, a sample of hOAT inactivated by 17 was denatured, releasing the cofactor and any other small molecules not covalently bound to the enzyme. Upon denaturation, the molecular mass of the native enzyme and inactivated enzyme were equivalent, demonstrating that the inactivator was not covalently bound to hOAT but instead had formed a tight-binding adduct, as also was indicated by the X-ray crystal structure. Thus, pathway h, in which Lys292 is covalently bound to the inactivator moiety to form a ternary adduct, was eliminated as a plausible mechanism of inactivation. However, due to the limited resolution of the intact protein MS, the exact molecular mass of the adduct could not be determined, and pathways a and c could not be differentiated by these methods.

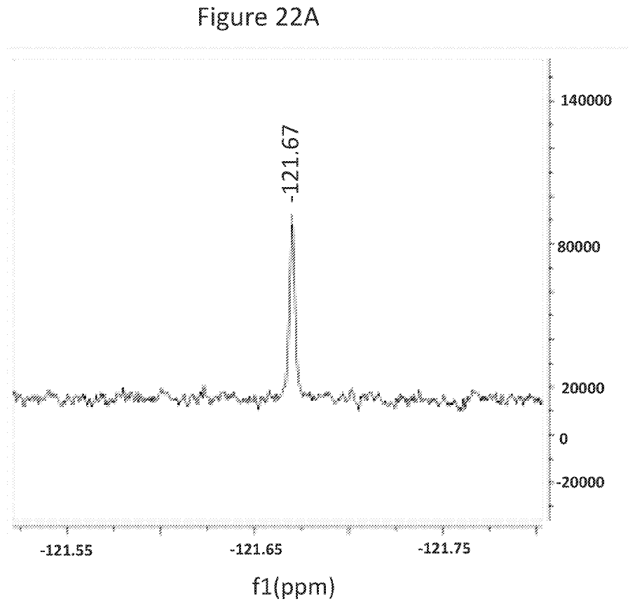

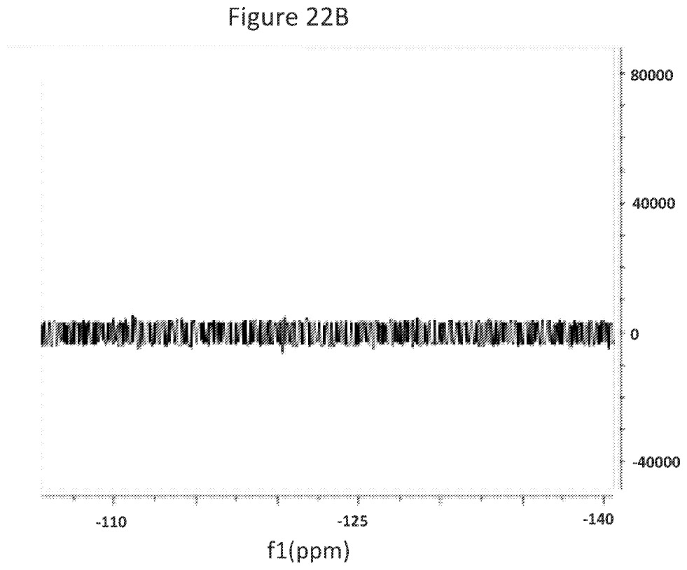

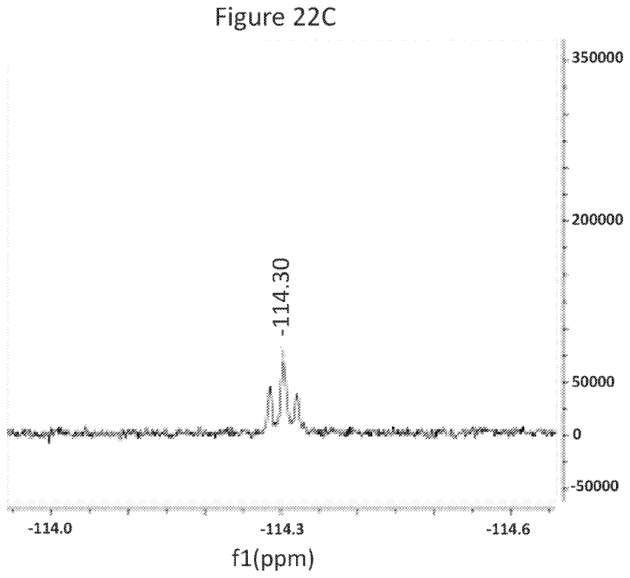

To determine if fluorine was contained in the inactivated enzyme adduct, ¹⁹F NMR spectroscopy was employed. The ¹⁹F NMR spectra of native hOAT, hOAT inactivated by 17 and dialyzed, and 17 inactivated hOAT denatured with formic acid were compared (FIG. 22A-22C). Trifluoroethanol was utilized as an internal standard. The NMR spectrum of the native enzyme showed no ¹⁹F peaks, demonstrating the expected absence of fluorine. The spectrum of the inactivated enzyme showed a single peak at −121.7 ppm, indicative of a single fluorine atom bound to the final adduct of the inactivated enzyme complex. When the enzyme was denatured, the peak shifted to −114.3 ppm (a 7.4 ppm shift downfield) and resolved into a doublet of doublets, coupled with the two adjacent protons. This suggests that the fluorine is more shielded in the enzyme pocket, likely the effect of weak electrostatic fields²⁹, which has been observed in other fluorinated protein ¹⁹F NMR studies. The change in the fluorine splitting is attributable to a broadening³⁰ in the peak due to the chemical shielding anisotropy contribution to the relaxation time of the nuclei. Collectively, these spectra suggest that an inactivated adduct bearing a single fluorine atom is the most consistent structure from inactivation of hOAT by 17. A fluorine atom was present in the active site, which was released upon denaturation, suggesting it was part of the PLP/inactivator complex. This indicates that pathway a, in which rapid aromatization occurs after the second deprotonation step, is most plausible.

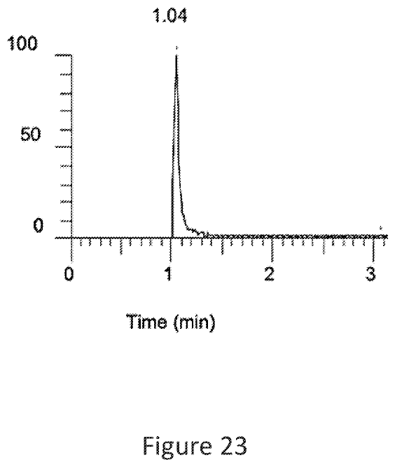

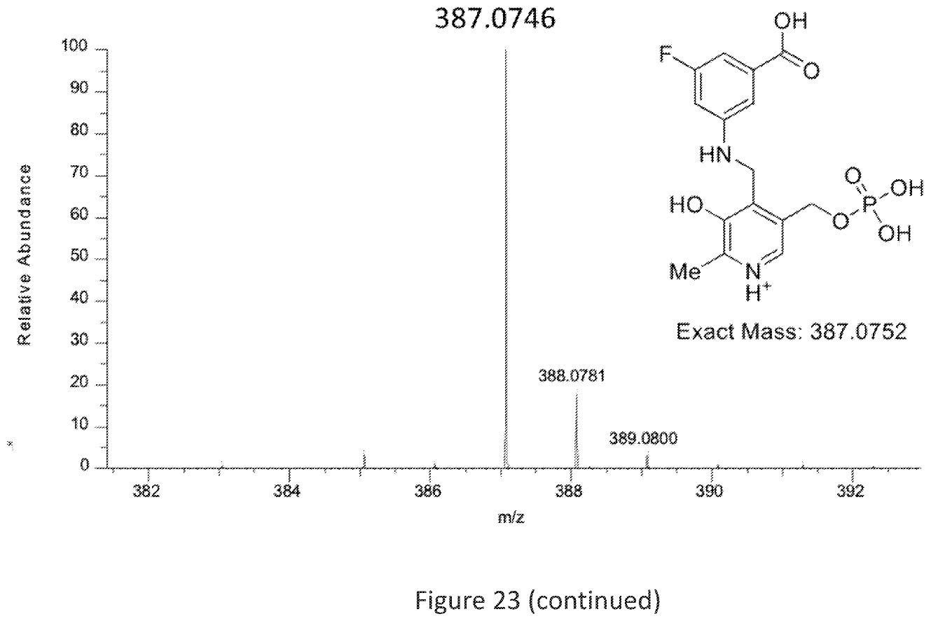

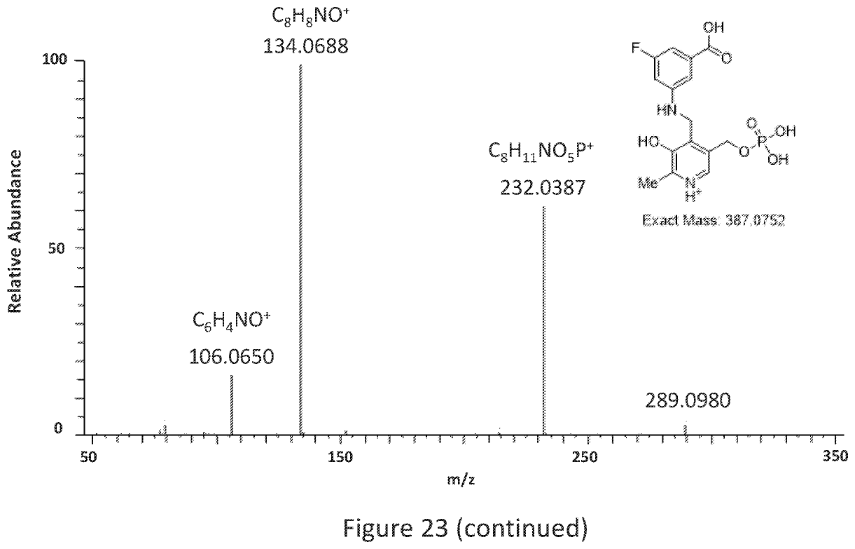

To further confirm these results, a sample of hOAT inactivated by 17 was denatured and the released small molecules were analyzed using LC-HRMS with alternating positive and negative mode electrospray to determine the molecular mass of the released PLP-17 complex. The major mass peak, observed in the correct range, was consistent with the PLP-17 adduct containing a single fluorine atom (obs: 387.0746, theoretical: 387.0752)(FIG. 23). The isotopic abundance of the parent ion as well as the spectra of MS/MS were consistent with elimination of one fluoride ion and rapid aromatization to give the PLP-17 adduct, further supporting inactivation pathway a, and eliminating pathway c.

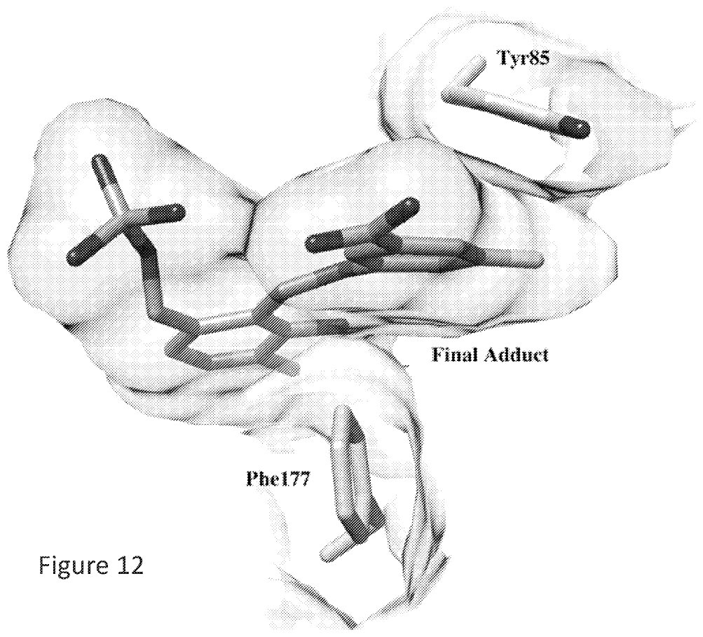

As seen in the crystal structure (FIG. 11), the remaining fluorine atom on the inactivator forms hydrophobic-like interactions with the side chain carbons of Glu235, aiding in the tight binding of the inactivator-PLP complex in the active site of hOAT.³¹ Although fluorine is able to form hydrogen bonds, there are no active site residues that reside within hydrogen bonding distance to the single fluorine atom of the inactivating adduct. Additionally, Tyr85 and Phe177 stabilize the pose of the inactivator aromatic ring, thus contributing to molecular recognition (FIG. 12).

Determination of the Turnover Mechanism to Metabolites The partition ratio of an MBI is the ratio of compound that acts as a substrate relative to the compound that inactivates the enzyme. Ideally, plotting the enzyme activity remaining after inactivation versus equivalents of compound added will provide a straight line from 100 to 0% enzyme activity remaining. The x-intercept of this line would give the number of inactivator molecules required to inactivate a single enzyme active site (the turnover number). This number represents the total number of compound molecules that were engaged by the active site until the enzyme was inactivated. Of these compounds, only the final one will inactivate the enzyme, and the others will have been consumed as substrates. As a result, the partition ratio is the turnover number minus one (assuming a 1:1 stoichiometry of inactivator to enzyme in the inactivated complex).

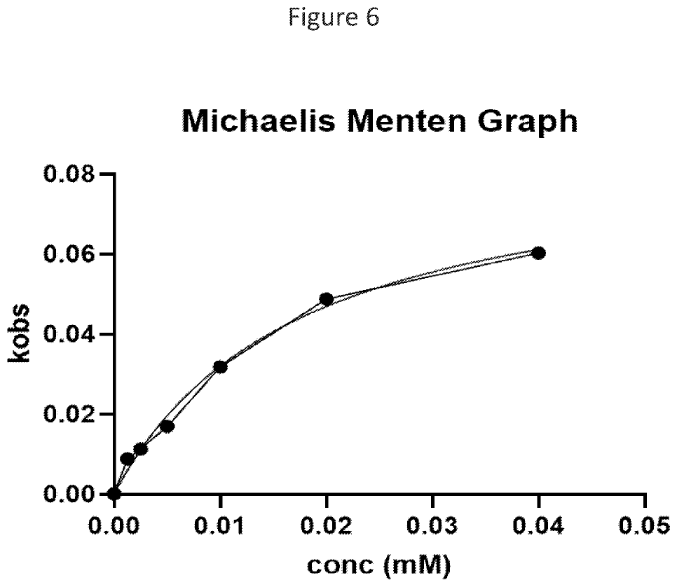

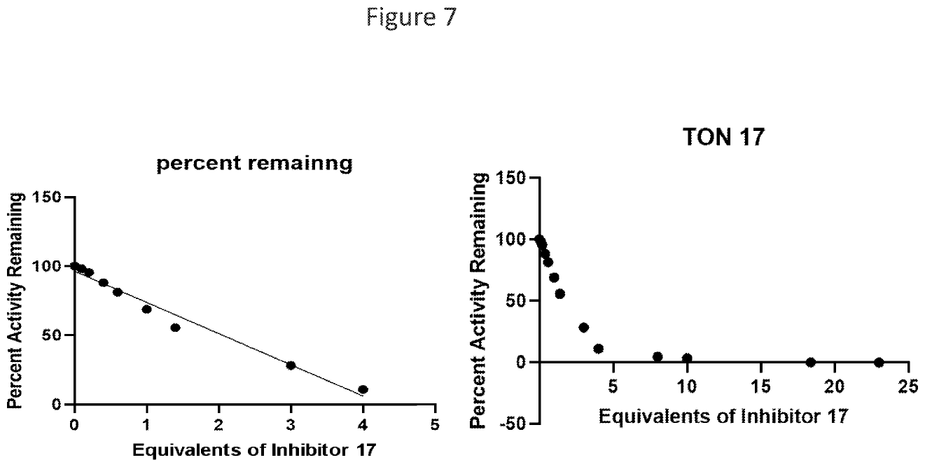

The partition ratio of 17 was determined by titrating a known amount of hOAT with varying equivalents of inactivator (FIG. 7). The linear relationship of the remaining enzymatic activity was extrapolated to yield the exact number of equivalents of 17 to inactivate the enzyme completely. From this we determined the turnover number to be 4.27 and thus the partition ratio of 17 to be 3.27.

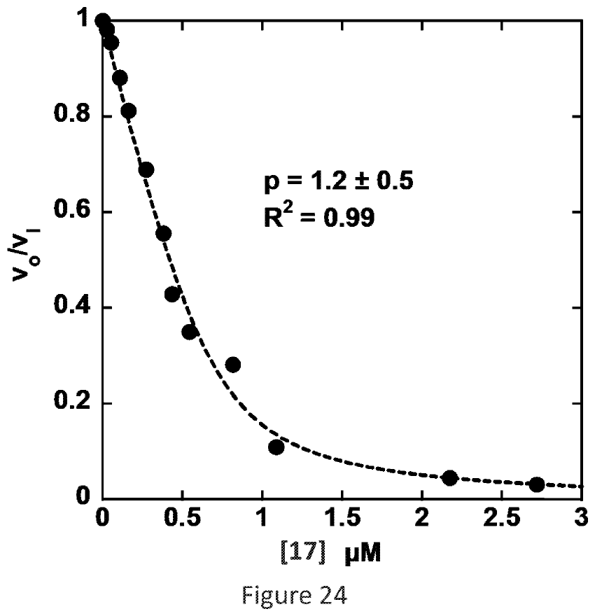

The turnover number for 17 was determined to be 2.2 by titrating a fixed amount of hOAT with varying equivalents of 17, measuring the remaining enzyme activities for the different equivalents of inhibitor, and employing the method of Pham et al.³² (FIG. 24). The partition ratio, therefore, is 1.2. The nonlinear part of the graph indicates deviation from pseudo-first order inactivation kinetics at higher concentrations of 17, and probably represents competition of the product with 17 for the active site.³³ A similar effect was observed in the inhibition of GABA-AT by the previously reported inactivator CPP-115.³⁴

Measuring the release of fluoride ions under different conditions can help determine if the turnover mechanism involves the formation of PMB or the release of PLP. In the absence of alpha-KG, if PMP is formed in the turnover mechanism it cannot be converted back to PLP. However, if the turnover mechanism releases PLP then no alpha-KG is required to allow multiple turnover events. In the presence of alpha-KG, regardless of turnover mechanism, the total number of fluoride ions released per active site can provide insight into the mechanism of the inactivation as well. The number of fluoride ions released per enzyme turnover can be detected using a fluoride ion selective electrode.

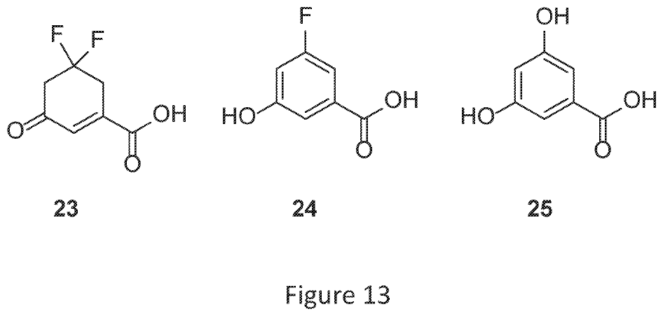

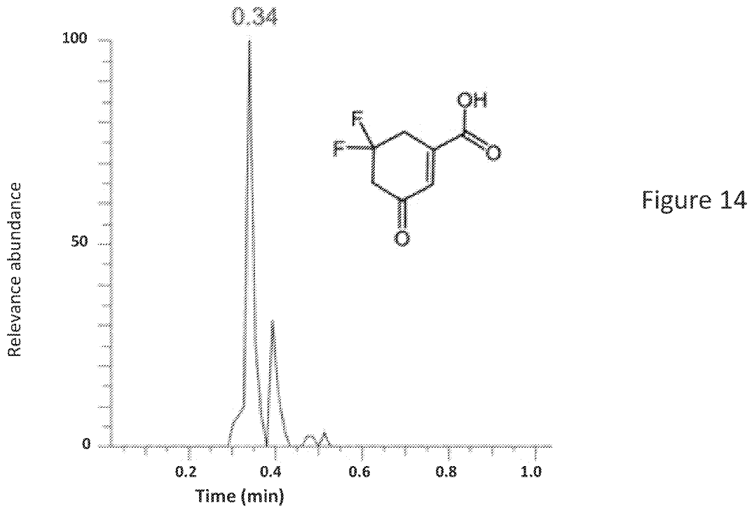

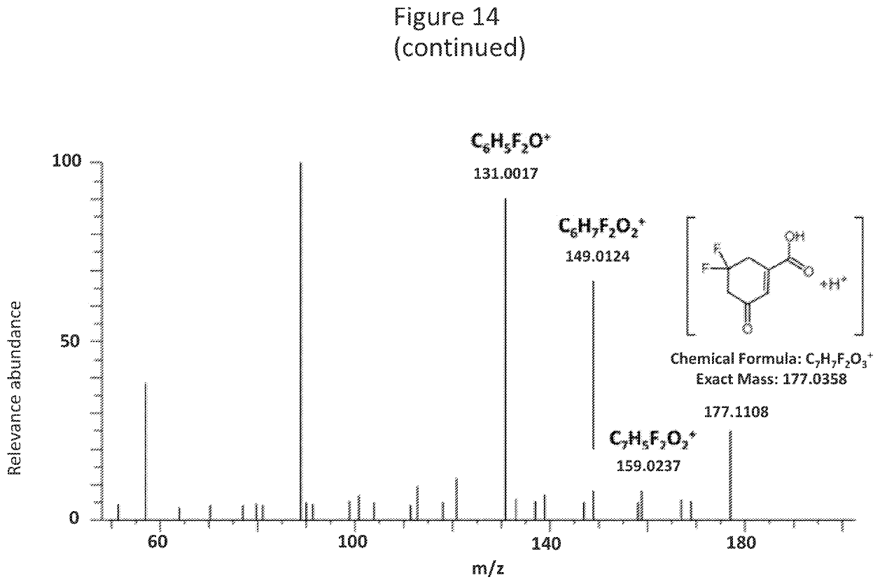

To identify turnover products produced during the inactivation of hOAT by 17, a sample of hOAT was inactivated in the presence of α-KG, which facilitates multiple turnover cycles and maximizes the yield of the turnover products. The sample was then filtered through a 10 kDa molecular weight cutoff spin filter to remove the protein. The flowthrough was collected, and the sample was then subjected to untargeted LC-HRMS with alternating positive and negative mode electrospray. Three possible turnover products were proposed, 23, 24, and 25 (FIG. 13). Among the potential metabolites, 23 was detected (observed 177.1108 m/z, [M+H]⁺, theoretical: 177.0358 m/z), and tandem mass spectrometry (MS/MS) showed fragmentation consistent with 23 (FIG. 14).

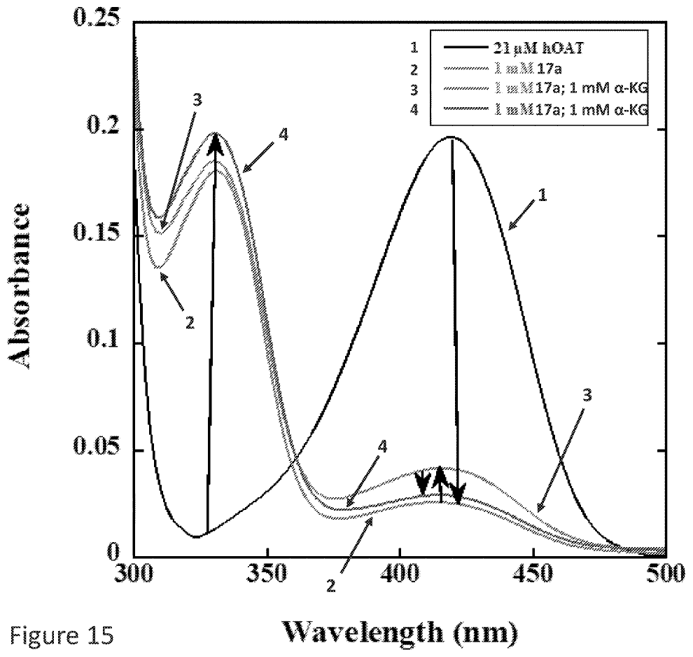

In addition to mass spectrometry, UV-vis spectrophotometry (FIG. 15) was utilized as a qualitative/confirmatory method to gather evidence for PMP formation resulting from turnover of the inactivator. An initial spectrum of the hOAT was collected (line 1), displaying a peak at 420 nm indicative of an internal aldimine. The enzyme was then treated with 1 mM of 17, and another spectrum was collected after two minutes (line 2). The decay of internal aldimine absorption peak at 420 nm with concomitant formation of a peak at 330 nm was observed. If PMP is produced upon turnover of the inactivator, the addition of α-KG is expected to result in reformation of a peak within the 410-420 nm range, characteristic of the PLP form of the cofactor. After hOAT was incubated with 17 for four minutes, 1 mM α-KG was added to the sample, and a third spectrum was immediately recorded (line 3). The observed reformation of a peak at 415 nm is indicative of transient accumulation of the external aldimine intermediate, assuming the binding of 17 is rapid; this observation further supports that PMP is likely produced upon turnover of 17 by hOAT. Two minutes after α-KG addition, a fourth spectrum was collected (line 4). The peak at 415 nm decayed, presumably resulted from subsequent engagement of residual 17 inactivating the PLP form of hOAT generated by the addition of α-KG. The steady accumulation at 330 nm is most likely a combination of the absorbance from PMP production and the final adduct of the inactivated enzyme.

Transient State Measurements of hOAT Inactivation by 17

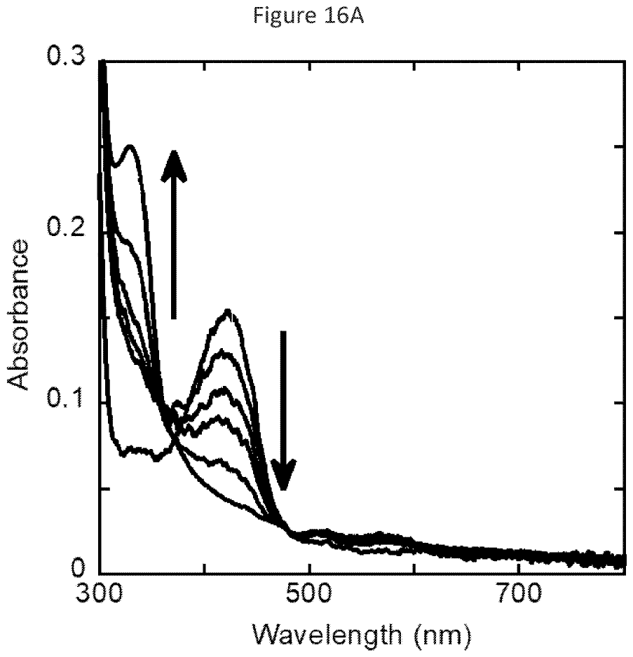

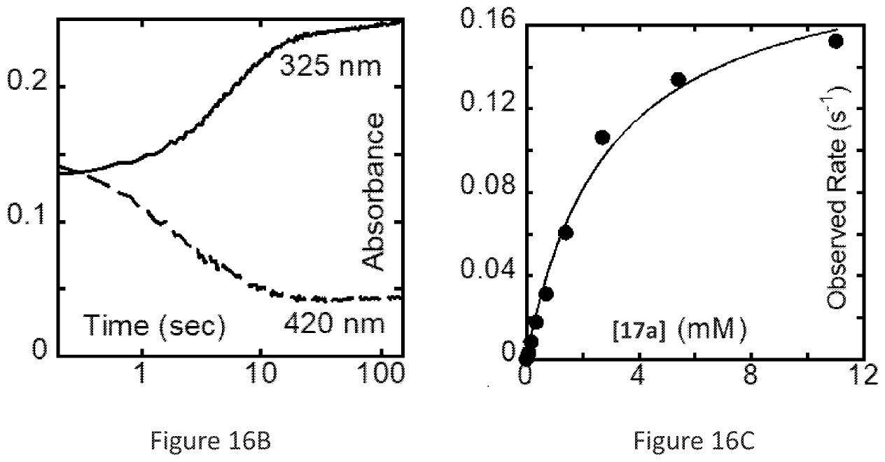

The data collected using stopped-flow spectrophotometry serves as additional evidence for the proposed mechanism. As can be seen from FIGS. 16A-16C, the extracted traces at each wavelength are ostensibly mirror images of each other and fit to return the same observed rate constant within error, indicating that there is likely only one phase that is observable spectrophotometrically. This single phase includes the near complete loss of the peak at 420 nm and the generation of a new peak at 325 nm. As shown above, the peak at 420 nm is present in the native enzyme and is common to enzymes containing the PLP cofactor. Diminishment of this peak is assigned as the loss of PLP as the initial adduct is formed. As described above, there are two possible explanations for the peak at 325 nm: formation of the final adduct and formation of PMP resulting from the deamination of 17 It has been shown that the addition of α-KG after an initial incubation period with 17 causes a temporary partial regeneration of the peak at 420 nm but does not appreciably diminish the peak at 325 nm (FIGS. 16A-16C). This indicates that the absorbance at 325 nm arises primarily from the final adduct instead of PMP; however, the absorbance at this wavelength likely contains contributions from both species.

These spectrophotometric data indicate that the rate limiting step of the processes are the same in the turnover and inactivation mechanisms, consistent with the idea that γ-proton abstraction from the initial external aldimine is rate limiting. The subsequent increase in absorbance at 325 nm would thus report the accumulation of both the turnover and inhibition products, with no difference in rates. In the absence of more spectrophotometric signals, it is impossible to assign intrinsic rates of specific steps; however, the dependence of the observed rate of decay accumulation of the absorption at 325 nm has a hyperbolic dependence, indicating that the dissociation constant for reversible formation of the external aldimine is 2.9±0.4 mM, consistent with 17 being a relatively bulky non-native ligand.

### Discussion

Plausible Mechanisms for 17: Based on all obtained results, the plausible inactivation mechanism is shown in Scheme 3, which commences with the condensation of inactivator 17 with Lys292-PLP complex 23, displacing the lysine and producing the external aldimine intermediate (M1). Intermediate M1 is then deprotonated by Lys292 at the γ-position, followed by a rapid tautomerization to quinonoid intermediate 28, resulting in formation of the ketimine intermediate (M2). A second deprotonation by Lys292 at the δ-position of M2 produces activated intermediate 30, which rapidly aromatizes to the final tight-binding adduct (31). This is the structure that was captured in the crystal structure of the inactivated enzyme (FIG. 11). Based on the turnover number/partition ratio study (FIG. 24), turnover results in inactivation 45% of the time (once for three turnovers). In addition to the inactivation pathway, 55% of 17 undergoes turnover to a product, 29 and PMP. The structure of the turnover product was supported by LC-MS and confirmed by its fragmentation spectrum (FIG. 14). The use of UV-vis spectrophotometry confirmed PMP production upon turnover of the inactivator (FIG. 15).

Scheme 3. Plausible Mechanism of Inactivation of hOAT by 17.

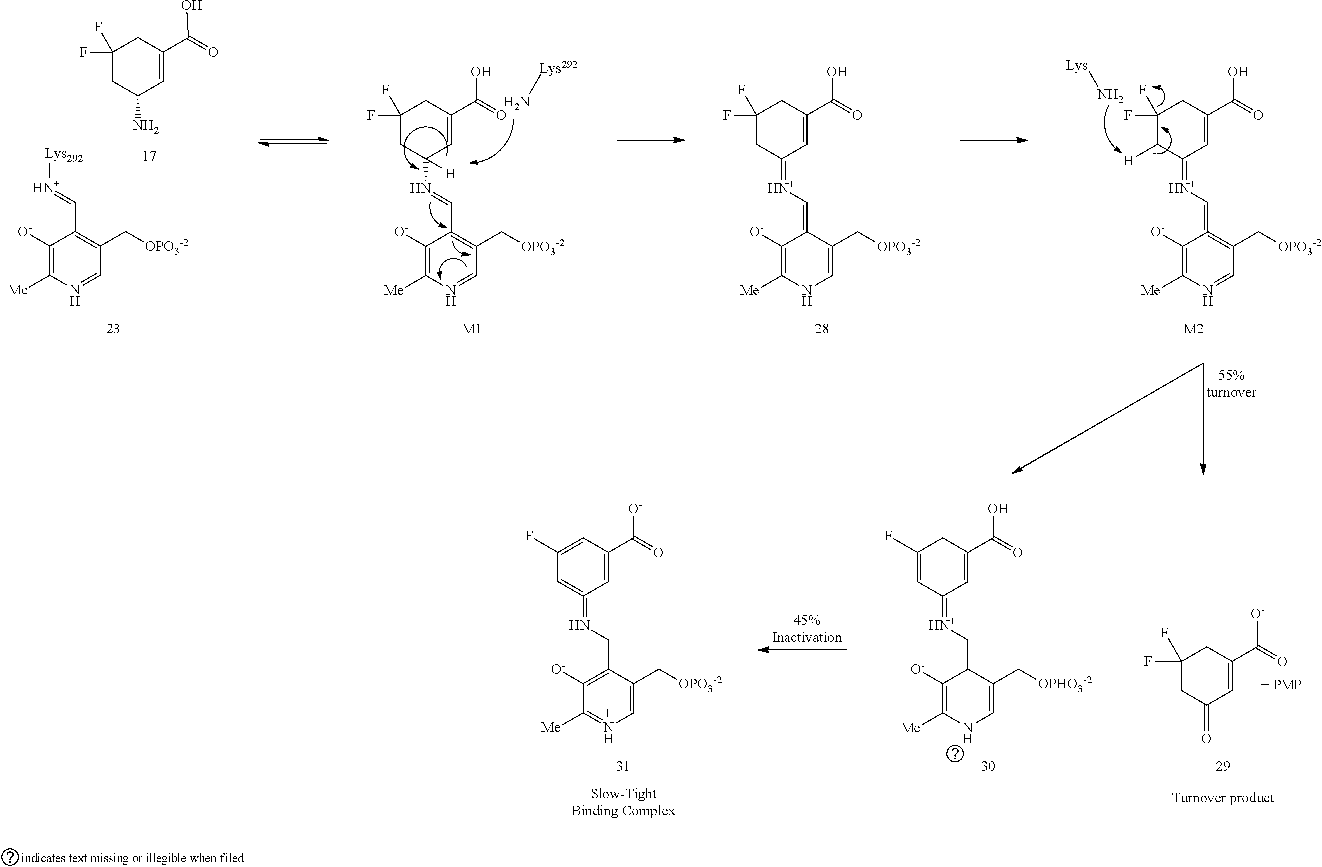

### Conclusion

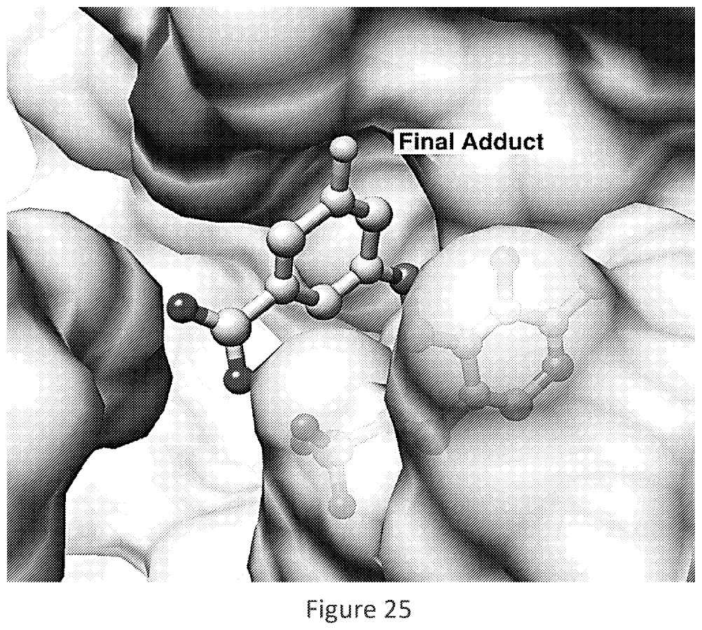

In recent years, the selective inactivation of human ornithine aminotransferase (hOAT) has been of significant interest as a potential therapeutic approach to halt the progression of hepatocellular carcinoma (HCC).¹⁹ In this work, a novel series of fluorinated cyclohexene analogues was rationally designed, synthesized, and evaluated for the inactivation of hOAT. Of the potential inactivators, 17 was demonstrated to be a selective inactivator of hOAT, resulting in a tight-binding adduct. A plausible mechanism of inactivation of hOAT by 17 is proposed (Scheme 3), which was elucidated using a variety of experimental methods, including X-ray crystallography, ¹⁹F NMR spectroscopy, mass spectrometry, and stopped-flow spectrophotometry. A previously study on the mechanism of inactivation of hOAT by (S)-3-amino-4,4-difluorocyclohex-1-ene-1-carboxylic acid concluded that, for this MBI, nucleophilic attack by Lys292 leads to formation of a ternary final adduct.³⁵ interestingly, in the inactivation mechanism of 17 with hOAT, despite the enhanced electrophilicity at C_{ε} of 30 conferred by the remaining fluorine atom, Lys292 did not attack the inactivator-PLP complex to form a ternary adduct. In the binding pocket, the position of the electrophilic center at C_{ε} is observed as distal from Lys292, likely accounting for the lack of Lys292 nucleophilic attack on 30. In addition, although structural evidence indicates the inactivated adduct is solvent accessible (FIG. 25), no water molecule was involved in the inactivation mechanism of hOAT, only the turnover mechanism. We believe this may be due to the lack of active site residues proximal to C_{ε} that could activate and assist water molecules for nucleophilic attack. This differs from the inactivation mechanisms of GABA-AT by previously reported potent inactivators CPP-115³⁴ and vigabatrin³⁶ and emphasizes the role of active site differences between hOAT and GABA-AT. Collectively, these findings provide insight for the future rational design of mechanism-based inactivators of hOAT. Experimental

All chemicals were purchased from Sigma Aldrich, Acros Organics, Fisher Scientific, or Combi-blocks and used without further purification. Anhydrous solvents (THF, CH₃CN, DMF) were purified before use by passing through a column composed of activated alumina and a supported copper redox catalyst. Yields refer to chromatographically homogeneous materials. Flash column chromatography was performed on a Teledyne Combiflash Rf Plus automated flash purification system with various Teledyne cartridges (4-80 g, 40-63 μm, 60 Å). Purifications were performed with hexanes and ethyl acetate unless otherwise noted. Preparatory column chromatography was performed on a Teledyne ACCQPrep HP150 using a Phenomenex Luna Omega Polar C18 preparatory HPLC column (150×21.2 mm, 5 μm) with water/MeCN with 0.1% formic acid as the solvent system. ¹H and ¹³C NMR spectra were recorded on a Bruker Avance-III NMR spectrometer at 500 MHz and 126 MHz, respectively, in CDCl₃, CD₃OD or DMSO-d6. ¹⁹F NMR spectra were recorded on a 400 MHz Bruker Avance III HD Nanobay system. Chemical shifts were reported in ppm; multiplicities are indicated by s=singlet, brs=broad singlet, d=doublet, t=triplet, q=quartet, dd=doublet of doublet, dt=doublet of triplet, dq=doublet of quartet, m=multiplet resonance. Coupling constants ‘J’ were reported in Hz.

High-resolution mass spectral data were obtained on an Agilent 6210 LC-TOF spectrometer in the positive ion mode using electrospray ionization with an Agilent G1312A HPLC pump and an Agilent G1367B autoinjector at the Integrated Molecular Structure Education and Research Center (IMSERC), Northwestern University. Analytical HPLC was performed using a reversed-phase Agilent Infinity 1260 HPLC with a Phenomenex Kintex C-18 column (50×2.1 mm, 2.6 μm), detecting with UV absorbance at 254 nm. All final products were shown to be >95% pure by HPLC.

### Synthesis

Compound 5 was prepared in accordance with literature procedures.

N-((1R,3S,4R,5R)-4-Bromo-7-oxo-6-oxabicyclo[3.2.1]octan-3-yl)acetamide (6). To a stirred solution of N-bromoacetamide (4.0 g, 29.00 mmol, 1.2 equiv) in dry CH₃CN (100 mL) in dark was added SnCl₄ (1 M in DCM, 9.67 mL, 9.67 mmol, 0.4 equiv) and water (0.522 mL, 29.00 mmol, 1.2 equiv) at r.t. After 5 min, to this solution was added 5 (3.0 g, 24.17 mmol, 1.0 equiv) in dry CH₃CN (20 mL). The reaction was stirred at r.t. overnight in dark. After the completion of the reaction was indicated by TLC (hexane:EtOAc=2.1), the reaction was quenched with aqueous Na₂SO₃ (50 mL) and aqueous NaHCO₃ (50 mL). The mixture was extracted with DCM (150 mL) for 3 times. The organic phase was separated, combined, washed with brine (150 mL), and dried with anhydrous Na₂SO₄. The solution was concentrated and diluted with EtOAc (30 mL). The suspension was stirred at r.t. for 2 h before the filtration. The white solid was collected and dried (6, 4.235 g, 67%). ¹H NMR (500 MHz, CDCl₃) δ 6.09 (d, J=4.8 Hz, 1H), 4.91 (dd, J=5.8, 4.0 Hz, 1H), 4.59 (t, J=72 Hz, 1H), 4.26 (dd, J=3.7, 1.3 Hz, 1H), 2.68-2.64 (m, 2H), 2.39 (ddd, J=14.7, 6.6, 2.9 Hz, 2H), 2.02 (dtd, J=14.7, 3.7, 1.7 Hz, 1H), 1.95 (s, 3H). ¹³C NMR (126 MHz, CDCl₃) δ 178.6, 169.2, 79.5, 50.1, 45.9, 35.8, 31.9, 30.1, 23.3. LRMS [M+H]⁺: 261.9.

N-((1R,3S,5R)-7-oxo-6-oxabicyclo[3.2.1]octan-3-yl)acetamide (7). To a solution of 6 (25.05 g, 95.6 mmol, 1.0 equiv) in benzene (774 mL) was added AIBN (1.57 g, 9.6 mmol, 0.1 equiv) and Bu₃SnH (30.9 mL, 114.7 mmol, 1.2 equiv). The solution was degassed then refluxed for 12 h. The solution was allowed to cool to room temperature before being concentrated under reduced pressure. To the resulting oil was added diethyl ether (200 mL), and the resulting solution was stirred for an hour during which time a white solid formed. The solution was filtered and the precipitate was washed with diethyl ether. The resulting white solid was purified by silica gel chromatography (0-30% MeOH in DC M) to afford 7 as a white solid (14.7 g, 84% yield). ¹H NMR (500 MHz, CDCl₃) δ 6.07 (s, 1H), 4.93-4.87 (m, 1H) 4.43-4.36 (m, 1H), 2.59 (q, J=4.3, 3.8 Hz, 1H), 2.46 (dt, J=11.5, 5.4 Hz, 1H), 2.18 (d, J=13.4 Hz, 1H), 2.04 (dd, J=4.3, 2.2 Hz, 2H), 1.96 (ddd, J=14.5, 6.1, 2.8 Hz, 1H), 1.90 (s, 3H), 1.79 (d, J=11.7 Hz, 1H). ¹³C NMR (126 MHz, CDCl₃) δ 179.29, 169.35, 77.48, 42.31, 36.84, 36 15, 33.70, 33.56, 23.46. HRMS (ESI) calc'd for C9H13NO3Na (M+Na⁺): 206.0793, found: 206.080.

Methyl (1R,3S,5R)-3-acetamido-5-hydroxycyclohexane-1-carboxylate (8). To 7 (8.6 g, 46.8 mmol, 1 equiv) at 0° C. was added 3.0 M HCl in methanol (40 mL) slowly. The resulting solution was then heated to 40° C. and stirred for 18 h until the starting material was consumed by LCMS. The reaction was concentrated to afford 7 as a white solid (10.1 g, 100%). ¹H NMR (500 MHz, Methanol-d₄) δ 3.72 (tt, J=12.1, 3.9 Hz, 1H), 3.60 (s, 3H), 3.56 (dt, J=11.1, 4.2 Hz, 1H), 2.43 (tt, J=12.7, 3.5 Hz, 1H), 2.13-1.98 (m, 3H), 1.95 (s, 3H), 1.23-1.07 (m, 3H). ¹³C NMR (126 MHz, MeOD) δ 174.75, 174.72, 67.11, 51.00, 48.45, 39.93, 39.00, 36.59, 33.28, 20.31. HRMS (ESI) calc'd for C10H17NO4Na (M+Na⁺): 238.1055, found: 238.104.

methyl (1R,3S)-3-acetamido-5-oxocyclohexane-1-carboxylate (9). To a solution of 8 (2.4 g, 11.4 mmol) in DCM (80 mL) was added PCC (7.3 g, 34.2 mmol, 3.0 equiv). The resulting solution was stirred overnight at room temperature. Upon completion, the reaction was quenched by addition of 30 mL sat. aq. Na₂S₂O₃ and allowed to stir for an hour until the brown solution turned a light green. The reaction was then filtered and acidified with 1 M HCl (4 mL). This mixture was diluted with EtOAc and the organic layer collected. The aqueous layer was extracted with EtOAc (6×40 mL). The organic layers were combined and washed with saturated brine (50 mL) and dried over Na₂SO₄, filtered, and concentrated to afford 9 as a white solid (1.6 g, 65%) ¹H NMR (500 MHz, CDCl₃) δ 4.21 (dtd, J=11.4, 7.2, 3.9 Hz, 1H), 3.72 (s, 3H), 2.88-2.79 (m, 1H), 2.79-2.70 (m, 1H), 2.63 (ddt, J=14.9, 4.9, 1.7 Hz, 1H), 2.55-2.38 (m, 2H), 2.25 (ddd, J=14.4, 11.5, 1.1 Hz, 1H), 1.99 (s, 3H), 1.75 (dt, J=13.1, 11.2 Hz, 11H). ¹³C NMR (126 MHz, CDCl₃) δ 205.6, 173.8, 169.4, 52.4, 47.0, 46.9, 42.4, 39.3, 33.8, 23.4. HRMS (ESI) calc'd for C10H15NO4Na (M+Na⁺): 236.0898, found: 236.090.

Methyl (1R,5S)-5-acetamido-3,3-difluorocyclohexane-1-carboxylate (10) and methyl (1S,5R)-5-acetamido-3-fluorocyclohex-3-ene-1-carboxylate (11) To a solution of 9 (2.7 g, 13.1 mmol, 1.0 equiv) in DCM (80 mL) in a plastic RBG was added DAST (5.0 mL, 38.0 mmol, 3.0 equiv) at 0° C. The solution was allowed to warm to room temp and then heated to 50° C. for 5 h. The reaction was then allowed to cool to room temperature and was quenched by addition of 20 mL sat. aq. NaHCO₃. This mixture was diluted with DCM and the organic layer collected. The aqueous layer was extracted with DCM (2×20 mL). The organic layers were combined and washed with saturated brine (20 mL) and dried over Na₂SO₄, filtered, and concentrated. The resulting residue was purified by silica gel chromatography (0-100% EtOAc in hexane) to afford a mixture of 10 and 11 as a white solid (2.31 g) which was carried forward without further purification.

Methyl (5S)-3,3-difluoro-5-(2,2,2-trifluoroacetamido)cyclohexane-1-carboxylate (14). To a 40 mL vial equipped with a pressure relief lid was added a mixture of 10 and 11 (3.21 g, ~13 mmol, 1.0 equiv) followed by 60 mL of 3M HCl in methanol. The vial was heated to 75° C.: for 2 days. After which, the solution was allowed to cool to room temperature, concentrated, and azeotroped with toluene. The residue was then suspended in DCM (20 mL) and TFAA (3 mL) and allowed to stir overnight at room temperature. The reaction was then concentrated, and the resulting residue was purified by silica gel chromatography (0-40% EtOAc in hexane) to afford 14 as a white solid (945 mg, 25% from 9). ¹H NMR (500 MHz, CDCl₃) δ 6.63 (s, 1H), 4.18 (tdt, J=11.5, 8.6, 4.4 Hz, 1H), 3.73 (s, 3H), 2.81 (tt, J=11.1, 4.2 Hz, 1H), 2.50-2.30 (m, 3H), 2.06-1.76 (m, 2H), 1.59 (q, J=12.2 Hz, 1H). ¹³C NMR (126 MHz, CDCl₃) δ 173.42, 156.54 (d, J=37.4 Hz), 124.90-119.41 (in), 115.59 (d, J=287.9 Hz), 52.53, 45.44 (d, J=10.1 Hz), 38.50 (t, J=24.6 Hz), 37.55 (d, J=9.4 Hz), 35.30 (t, J=25.0 Hz), 32.14. HRMS (ESI) calc'd for C10H11F5NO3 (M−H): 288.0659, found: 288.067.

Methyl (S)-3,3-difluoro-5-(2,2,2-trifluoroacetamido)cyclohex-1-ene-1-carboxylate (15) and methyl (R)-5,5-difluoro-3-(2,2,2-trifluoroacetamido)cyclohex-1-ene-1-carboxylate (16). To a solution 1 M KHMDS (7.85 mL, 7.85 mmol, 2.4 equiv) in dry THF (10 mL) at −78° C. was added a solution of 14 (945 mg, 3.3 mmol, 1.0 equiv) in THF dropwise over 20 minutes. The solution was allowed to stir for 3 h at −78° C., after which a solution of PhSeBr (900 mg, 2.0 mmol, 1.4 equiv) in THF. The reaction was allowed to warm to room temperature overnight, after which it was quenched by addition of 15 mL sat. aq. NH₄Cl. This mixture was diluted with EtOAc and the organic layer collected. The aqueous layer was extracted with EtOAc (2×10 mL). The organic layers were combined and washed with saturated brine 105 mL) and dried over Na₂SO₄, filtered, and concentrated. The resulting residue was purified by silica gel plug (0-100% EtOAc in hexane) and the resulting light yellow oil was carried on crude.

To a solution of the yellow oil in DCM (5 mL) was added mCPBA (820 mg, 3.3 mmol, 1.5 equiv) and the reaction was allowed to stir for 3 h at room temperature. The reaction was quenched by the addition of 10 mL sat. aq. Na₂S₂O₃, diluted with DCM, and the organic layer collected. The aqueous layer was extracted with DCM (2×10 mL). The organic layers were combined and washed with sat. aq. NaHCO₃ (10 mL), saturated brine (10 mL), and dried over Na₂SO₄, filtered, and concentrated. The resulting residue was purified by silica gel plug (0-20% MeOH in DCM), to provide a 1:1 mixture of 15 and 16 (534.5 ng, 56% over two steps). Purification by C18 silica gel chromatography (10-90% MeCN in water) 15 (230 mg, 24.5% over two steps) and 16 (220 mg, 23.4% over two steps) as white solids along with mixed fractions 15: ¹H NMR (500 MHz, CDCl₃) δ 6.84 (td, J=5.2, 2.5 Hz, 1H), 6.50 (s, 1H), 4.45 (dq, J=11.7, 5.6 Hz, 1H), 3.81 (s, 3H), 2.81 (dqd, J=18.0, 4.9, 2.0 Hz, 1H), 2.55-2.36 (m, 2H), 2.30 (qd, J=13.3, 8.5 Hz, 1H). HRMS (ESI) calc'd for C10H9F5NO3 (M−H). 286.0502, found: 286.051

16: ¹H NMR (500 MHz, CDCl₃) δ 6.85 (dt, J=4.3, 2.0 Hz, 1H), 6.48 (d, J=8.0 Hz, 1H), 5.03 (s, 1H), 3.79 (s, 3H), 2.96 (dddd, J=18.5, 12.4, 10.5, 1.8 Hz, 1H), 2.83-2.69 (m, 1H), 2.44-2.30 (m, 1H). HRMS (ESI) calc'd for C10H9F5NO3 (M−H), 286.0502, found: 286.051

### (R)-3-amino-5,5-difluorocyclohex-1-ene-1-carboxylic acid hydrochloride (17)

To a 40 mL vial equipped with a pressure relief lid was added 16 (215 mg, 0.75 mmol) and 4 M HCl aq at 80 C until the starting material and monodeprotected intermediates were consumed. The reaction was then concentrated and azeotropde with acetonitrile (3×20 mL). The material was purified via a C18 aqueous column (0-40% MeCN in water) to yield 17 as a tan solid (70.4 mg, 43% yield). ¹H NMR (500 MHz, D₂O) δ 6.83 (s, 1H), 4.34 (d, J=7.6 Hz, 1H), 2.97-2.80 (m, 2H), 2.59 (ddt, J=19.9, 12.9, 6.0 Hz, 1H), 2.31-2.17 (m, 1H). ¹³C NMR (126 MHz, D₂O) δ 168.42, 131.81, 131.01, 121.70 (d, J=239.2 Hz), 46.03, 33.18-32.63 (m). ¹⁹F NMR (470 MHz, D₂O) δ −93.69 (d, J=247.2 Hz), −95.90 (d, J=247.1 Hz). HRMS (ESI) calc'd for C7H10F2NO2 (M+H⁺): 178.0679, found: 178.068

### (S)-5-amino-3,3-difluorocyclohex-1-ene-1-carboxylic acid hydrochloride (17a)

17a was prepared from 15 (50 mg, 0.18 mmol) via the same procedure as 17. 17a was isolated as a tan solid (33 mg, 87% yield) Characterization: ¹H NMR (500 MHz, D₂O) δ 6.96-6.85 (m, 1H), 3.92 (tt, J=9.1, 4.1 Hz, 1H), 3.04 (dd, J=18.2, 6.2 Hz, 1H), 2.77 (tdt, J=13.1, 8.6, 4.3 Hz, 1H), 2.60 (ddt, J=17.9, 9.1, 2.9 Hz, 1H), 2.47 (dddd, J=22.5, 13.7, 11.1, 9.2 Hz, 1H). ¹³C NMR (126 MHz, D₂O) δ 168.40, 136.09 (t, J=10.5 Hz), 127.75 (dd, J=31.6, 28.4 Hz), 117.86 (t, J=233.1 Hz), 43.55 (d, J=8.1 Hz), 34.11 (t, J=25.7 Hz), 27.31. 19F NMR (470 MHz, D₂O) δ −83.62-−86.13 (m), −91.33 (dq, J=284.2, 9.0 Hz). HRMS (ESI) calc'd for C7H10F2NO2 (M+H⁺): 178.0679, found: 178.068.

Methyl cis-3-((tert-butoxycarbonyl)amino)-5-hydroxycyclohexane-1-carboxylate (19). To a stirred solution of 3-amino-5-hydroxybenzoic acid hydrochloride (18) (4.0 g, 21.1 mmol, 1.0 equiv) in water (10 mL) was added Rh/Al₂O₃ (15% wt, 150 mg). The suspension was charged with high pressure H₂ (400 psi), heated to 90° C. and stirred overnight. After the completion of reaction was detected by LC-MS, the suspension was filtered. The obtained filtrate was concentrated and then diluted with MeOH (20 mL). The solution was cooled to 0° C. with an ice bath, followed by slow addition of SOCl₂ (4.65 mL, 63.29 mmol, 3.0 equiv) under Ar. The solution was warmed to r.t. and stirred for 1 h. After the completion of reaction was detected by LC-MS, the solution was concentrated and diluted with MeOH (20 mL). To this stirred solution was added Boc₂O (6.91 g, 31.65 mmol, 1.5 equiv) and NaHCO₃ (5.32 g, 63.29 mmol, 3.0 equiv). The suspension was stirred at r.t. overnight before being filtered. The filtrate was concentrated and purified by silica gel chromatography (50-100% EtOAc in hexane) to afford a white solid (19, 2.13 g, 37%). ¹H NMR (500 MHz, CDCl₃) δ 4.54 (brs, 1H), 3.76-3.69 (m, 1H), 3.68 (s, 3H), 3.59-3.48 (m, 1H), 2.43 (tt, J=12.2, 3.6 Hz, 1H), 2.28-2.15 (m, 3H), 1.86 (d, J=4.5 Hz, 1H), 1.43 (s, 9H), 1.37 (q, J=12.3 Hz, 1H), 1.23 (q, J=12.2 Hz, 1H), 1.13 (q, J=11.4 Hz, 1H). ¹³C NMR (126 MHz, CDCl₃) δ 174.8, 155.2, 79.7, 68.3, 52.1, 47.2, 41.7, 39.3, 37.0, 34.8, 28.5. LRMS [M+H]⁺: 274.0, calc'd for C₃H₂₄NO₅ (M+H⁺): 274.2.

Methyl cis-3-((tert-butoxycarbonyl)amino)-5-oxocyclohexane-1-carboxylate (20). To a stirred solution of 19 (2.13 g, 7.79 mmol, 1.0 equiv) in DCM (100 mL) was added PCC (5.04 g, 23.38 mmol, 3.0 equiv). The suspension was stirred at r.t. overnight. The reaction was filtered, and the obtained filtrate was concentrated and purified by silica gel chromatography (0-20% EtOAc in DCM) to afford a white solid (20, 1.68 g, 80%). ¹H NMR (500 MHz, CDCl₃) δ 4.58 (brs, 1H), 3.84 (brs, 1H), 3.71 (s, 3H), 2.78-2.69 (m, 2H), 2.59 (ddt, J=14.9, 4.6, 1.8 Hz, 1H), 2.51-2.43 (m, 2H), 2.28-2.16 (m, 1H), 1.79-1.66 (m, 1H), 1.44 (s, 9H). ¹³C NMR (126 MHz, CDCl₃) δ 205.9, 173.8, 154.7, 80.1, 52.5, 48.3, 47.7, 42.6, 39.4, 34.7, 28.5. LRMS [M+H]⁺: 272.1, calc'd for C₁₃H₂₂NO₅ (M+H⁺): 272.1.

Methyl cis-5-((tert-butoxycarbonyl)amino)-3,3-difluorocyclohexane-1-carboxylate (21). To a stirred solution of 20 (700 mg, 2.58 mmol, 1.0 equiv) in 1, 2 dichloroethane (30 mL) was added DAST (1.36 mL, 10.32 mmol, 3.0 equiv). The solution was heated to 40° C. and stirred overnight. The completion of the reaction was indicated by TLC (hexane:EtOAc=2:1). The reaction was quenched with aqueous NaHCO₃ (20 mL) and extracted with DCM (30 mL) for 3 times. The organic phase was separated, combined, washed with brine (50 mL), and dried with anhydrous Na₂SO₄. The solution was concentrated and purified by silica gel chromatography (0-10% EtOAc in hexane) to afford a white solid (21, 448 mg, 59%). ¹H NMR (500 MHz, CDCl₃) δ 4.46 (s, 1H), 3.77 (s, 1H), 3.70 (s, 3H), 2.68 (ttd, J=12.8, 3.8, 1.6 Hz, 1H), 2.49-2.30 (m, 3H), 1.80 (dtd, J=34.2, 13.4, 3.3 Hz, 1H), 1.44 (s, 10H), 1.29 (q, J=12.6 Hz, 1H). ¹³C NMR (126 MHz, CDCl₃) δ 173.4, 154.7, 122.2 (dd, J=244.1, 239.2 Hz), 79.9, 52.2, 46.0, 40.0 (t, J=23.7 Hz), 37.8 (d, J=10.8 Hz), 35.5 (t, J=25.1 Hz), 34.3 (d, J=1.6 Hz), 28.3. LRMS [M+H]⁺: 294.0, calc'd for C₁₃H₂₃F₂NO₄ (M+H⁺): 294.1.

cis-5-Amino-3,3-difluorocyclohexane-1-carboxylic acid hydrochloride (22). Following the same procedure as 17.21 (50 mg) was converted to 22 (21 mg, 59%) as a white solid. ¹H NMR (500 MHz, CD₃OD) δ 3.41 (tt, J=12.1, 4.3 Hz, 1H), 2.73 (ttd, J=12.9, 3.8, 1.7 Hz, 1H), 2.52-2.35 (m, 3H), 1.99-1.83 (m, 2H), 1.57 (q, J=12.5 Hz, 1H). ¹³C NMR (126 MHz, CD₃OD) S 173.9 (d, J=1.6 Hz), 121.6 (dd, J=243.7, 238.2 Hz), 46.3 (d, J=11.9 Hz), 37.0 (dd, J=16.4, 6.2 Hz), 36.8 (d, J=25.6 Hz), 34.7 (t, J=24.7 Hz), 30.9 (d, J=1.4 Hz). HRMS (ESI) (m/z) [M−H]⁻ calc'd for C₇H₁₀F₂NO₂=178.0685, found=178.0686.

Expression and Purification of hOAT

The expression and purification of hOAT was performed similarly to previously published protocols.³⁷ In brief, Escherichia coli BL21 (DE3) cells containing the pMAL-t-hOAT plasmid were incubated at 37° C. and shaken at 200 rpm in 1 L of Luria-Bertani (LB) medium containing 100 μg/mL ampicillin. Upon reaching an OD600 value of 0.7 absorbance units, the cell culture was incubated on ice for 60 minutes before induction. Expression of the MBP-t-OAT fusion protein was induced utilizing 400 μM isopropyl β-D-1-thiogalactopyranoside, and the culture was incubated at 18° C. while being shaken at 200 rpm for 17 hours. The cells were harvested by two periods of centrifugation at 12400 g for 20 minutes before being resuspended in buffer A containing 50 mM Tris-HCl, 200 mM NaCl, and 100 μM PLP, pH 7.4 The suspension was lysed with one 5-minute period of sonication and followed by an additional two 20-minute periods of centrifugation at 40,000 g to pellet cell debris. The resulting supernatant was loaded onto an amylose affinity column pre-equilibrated with buffer A. The column was washed with 35 mL of buffer A before the MBP-t-OAT fusion protein was eluted utilizing a 100 mL linear gradient of buffer A supplemented with 10 mM maltose. Individual 4 mL fractions containing the MBP-t-OAT fusion protein were combined. To remove the MBP tag, the protein sample was diluted to −45 mL with cleavage buffer containing 50 mM Tris, 100 mM NaCl, pH 8.0 and treated with tobacco etch virus protease for 12 hours at 10° C. with 60 rpm agitation. Following cleavage, a 50,000 molecular weight cutoff (MWCO) Amicon Ultra centrifugal filter device was utilized to concentrate the protein mixture until the sample volume was less than 5 mL. For further purification, the sample was loaded onto a HiLoad Superdex-200PG column (16 mm×600 mm) pre-equilibrated with buffer A and subjected to size exclusion chromatography. The 4 mL fractions containing untagged hOAT were collected and quantified utilizing an extinction coefficient at 280 nm of 60200 M-1 cm-1 and molecular weight of 48500 Daltons. Aliquots of ~1 mg/mL hOAT supplemented with 20% glycerol were frozen in liquid nitrogen and stored at −80° C.

Expression and Purification of hGABA-AT and PYCR

Recombinant human brain γ-aminobutyric acid aminotransferase (hGABA-AT) was expressed and purified according to literature procedures with slight modifications.²⁶ Briefly, pelleted Expi293 cells were resuspended into buffer A containing 50 mM K₄O₇P₂ pH 8.6, 300 mM NaCl, and 100 μM PLP. The suspension was lysed using four repeat cycles of sonication (10 s on, 10 s off, 20% amplitude) on ice followed by the addition of 12.5 mM MgCl, 125 μM CaCl, and 5 mg of lyophilized DNase I. The lysate incubated on ice for 60 minutes with periodic inversions followed by a two step centrifugation procedure to pellet cell debris. Step one included centrifugation at 18000 rpm for 60 minutes at 4° C. followed by the collection of supernatant and a secondary step at 20000 rpm for 45 minutes. The supernatant was collected, and the sample was loaded into a disposable 0.45 μm bottle-top vacuum filter system to remove remaining cell debris. The resulting sample was then loaded onto a Co²⁺ immobilized metal affinity chromatography column pre-equilibrated with buffer A. The column was washed with 40 mL of buffer A before hGABA-AT was eluted using a step-wise elution gradient of buffer A supplemented with 200 mM imidazole. The step-wise gradient totaled 5 steps of increasing imidazole concentration (1 mM, 2 mM, 5 mM, 100 mM, 200 mM). Individual 4 mL fractions containing hGABA-AT were combined and concentrated to a volume of 3 mL using a 30000 Da molecular weight cutoff (MWCO) Amicon Ultra centrifugal filter device. For further purification, the sample was loaded onto a HiLoad Superdex-200PG column (16 mm×600 mm) pre-equilibrated with buffer A and subjected to size exclusion chromatography at a flow rate of 0.25 mL/min. PYCR1 was purified according to previously published protocols.³⁸ Coupled enzyme assays for hGABA-AT, hOAT, Ala-AT, and Asp-AT were carried out according to previous procedures.²⁶

### Dialysis Assay

The dialysis experiment was conducted using previous protocols.^{26,38}

### Partition Ratio Experiment

The partition ratio was calculated using previous protocols.^{26,28}

### ¹⁹F NMR Spectroscopy Studies

After being fully inactivated by 17, a sample of hOAT was washed in distilled H2O and diluted to 10 μM and spiked with trifluoroethanol as an internal standard. The sample was run on a Bruker Neo 600 MHz system with a QCI (HFCN) Cryo Probe w/Z-Gradient at 298 K for 960 scans. Next, the same sample was denatured with addition of 10 equivalents of formic acid and the resulting solution heated to 55° C. for 10 minutes before being submitted to the same 19F NMR conditions.

### Crystallization of OAT Inactivated by 17, X-Ray Data Collection and Processing, and Model Building and Refinement

Following confirmed inactivation of hOAT with 17, the sample was transferred to a 50,000 molecular weight cutoff (MWCO) Amicon Ultra centrifugal filter device and exchanged into buffer containing 100 mM Tricine, 100 mM NaCl, pH 7.8. The sample was then concentrated to ~6 mg/mL. Crystals were grown for 7 days at room temperature utilizing the hanging-drop vapor diffusion method with each drop initially containing 2 uL of inactivated hOAT and 2 uL of well solution. The crystals with the best morphology appeared in the well solution containing 10% PEG 6000, 100 mM NaCl, 20% glycerol, and 100 mM Tricine pH 7.8. Following 7 days of growth, hOAT crystals with the best morphology were transferred into a cryo-protectant solution containing well solution supplemented with 30% glycerol prior to being frozen in liquid nitrogen. Monochromatic X-ray diffraction data were collected at the LS-CAT, Advanced Photon Source (APS), beamline 21-ID-D at Argonne National Laboratory (ANL, Argonne, IL). Data were collected at a wavelength of 1.127 Å and a temperature of 100 K using a Dectris Eiger 9M detector. Datasets were processed and analyzed with autoPROC.48 The structure of inactivated OAT was solved by molecular replacement utilizing PHASER49 in Phenix.50 The search model was the previously published structure of OAT (PDB code: 6V8C). The model building and refinement were performed utilizing Coot51 and Phenix, respectively, until the lowest possible Rfree/Rwork factor values were achieved. UCSF Chimera52 was used for the preparation of structural depiction figures. The structure factors and the refined model were deposited in Protein Data Bank with PDB ID: 8V9M.

### UV-Vis Spectrophotometry

A Shimadzu UV-2450 spectrophotometer was utilized to collect absorbance spectra from 700 to 250 nm at 20° C. The instrument was baselined with 1 mM 17 and 1 mM α-KG in reaction buffer containing 100 mM HEPES pH 7.5 and 200 mM NaCl. The initial concentration of hOAT was determined with A₂₈₀ measurement and the molar extinction coefficient, previously calculated with amino acid composition.

### Intact Protein Mass Spectrometry

The hOAT intact protein mass spectrometry samples were analyzed on an Orbitrap Eclipse (ThermoFischer Scientific) mass spectrometer as previously reported.³⁸

### Small Molecule Mass Spectrometry

Samples were injected onto a Kinetex 1.3 μM, 100 Å C18 column on an Agilent 1290 Infinity II UHPLC system at 0.3 mL/min, in line with a Thermo Q-Exactive mass spectrometer at a monotonously increasing gradient of Solvent B (95% ACN/5% H₂O/0.1% FA). Positive-mode ESI data were collected with the HESI source set to 3.6 kV, 10 sheath gas, 300° C. probe temperature and 320° C. inlet capillary temperature. Untargeted data-dependent and targeted MS/MS data were collected with HCD fragmentation with a normalized collisional energy, NCE, of 35. For untargeted data-dependent methods the instrument was run with the following MS1 parameters, automatic gain control (AGC)=2e5 charges, Resolution=35,000, maximum injection time=40 ms, scan range=100-1000 m/z. Tandem MS data were acquired in a data-dependent fashion on the five most abundant precursor ions with a 2 m/z precursor isolation window, a minimum AGC target of 6e3, and dynamic exclusion of previously fragmented precursors set to 4 s.

### Transient State Methods

Transient-State Kinetic Characterization of hOAT with 028: A sample of hOAT (17 μM) was mixed with 17 (0-11 mM) at 20° C. using a rapid mixing stopped-flow spectrophotometer (TgK Scientific) equipped with charge-coupled device (CCD) detection, allowing for the collection time-resolved spectra. The reaction was observed at all wavelengths between 300-600 nm for 150 seconds. A reconstructed time zero point was included in the data and was obtained by adding the spectrum of the enzyme and the spectrum of the inhibitor when measured separately. The data were fit to a single exponential term describing a one-step irreversible model. Single wavelength traces were extracted from the time resolved spectra datasets at the wavelengths of maximal absorbance in the initial and final spectra (425 and 320 nm, respectively) to provide a visual comparison of the time-dependence of the events at each wavelength.

### Docking Study

Docking models of ligands bound to hOAT and GABA-AT were developed utilizing the Molecular Operating Environment (MOE) computational suite's Builder utility^{39,40} according to previously reported protocol.²⁷

### Theoretical pKa Calculations

The theoretical pKₐ calculations were performed using previous protocols.²⁷

### REFERENCES

(1) Sayiner, M.; Golabi, P.; Younossi, Z. M. Disease Burden of Hepatocellular Carcinoma. A Global Perspective. Digestive Diseases and Sciences 2019, 64 (4), 910-917. DOI: 10.1007/s10620-019-05537-2. Personeni, N.; Rimassa, L. Hepatocellular Carcinoma: A Global Disease in Need of Individualized Treatment Strategies. Journal of Oncology Practice 2017, 13 (6), 368-369. DOI: 10.1200/JOP.2017.024604 (accessed 2023/01/13). Sherman, M.; Bruix, J.; Porayko, M.; Tran, T.; for the, A. P. G. C. Screening for hepatocellular carcinoma: The rationale for the American Association for the Study of Liver Diseases recommendations. Hepatology 2012, 56 (3), 793-796, https://doi.org/10.1002/hep.25869. DOI. https://doi.org/10.1002/hep.25869 (accessed 2023/01/13). Yang, J. D.; Roberts, L. R. Hepatocellular carcinoma: A global view. Nat Rev Gastroenterol Hepatol 2010, 7 (8), 448-458. DOI: 10.1038/nrgastro.2010.100 PubMed.
(2) She, W. H.; Chok, K. S. Strategies to increase the resectability of hepatocellular carcinoma. World J Hepatol 2015, 7(18), 2147-2154. DOI: 10.4254/wjh.v7.i18.2147 PubMed.
(3) Santopaolo, F.; Lenci, I.; Milana, M.; Manzia, T. M.; Baiocchi, L. Liver transplantation for hepatocellular carcinoma. Where do we stand? World J Gastroenterol 2019, 25 (21), 2591-2602. DOI: 10.3748/wjg.v25.i21.2591 PubMed. Rahimi, R. S.; Trotter, J. F. Liver transplantation for hepatocellular carcinoma: outcomes and treatment options for recurrence. Ann Gastroenterol 2015, 28 (3), 323-330. PubMed
(4) Mahale, P.; Shiels, M. S.; Lynch, C. F.; Chinnakotla, S.; Wong, L. L.; Hernandez, B. Y., Pawlish, K. S.; Li, J.; Alverson, G.; Schymura, M. J.; et al. The Impact of Liver Transplantation on Hepatocellular Carcinoma Mortality in the United States. Cancer Epidemiol Biomarkers Prev 2021, 30 (3), 513-520. DOI: 10.1158/1055-9965.EPI-20-1188 PubMed
(5) Sarveazad, A.; Agah, S.; Babahajian, A.; Amini, N.; Bahardoust, M. Predictors of 5 year survival rate in hepatocellular carcinoma patients. J Res Med Sci 2019, 24, 86-86. DOI: 10.4103/jrms.JRMS_1017_18 PubMed.
   (6) Pasini, F.; Serenari, M.; Cucchetti, A.; Ercolani, G. Treatment options for recurrence of hepatocellular carcinoma after surgical resection: review of the literature and current recommendations for management. Hepatoma Research 2020, 6, 26. DOI: 10.20517/2394-5079.2019.47. Saito, A.; Toyoda, H.; Kobayashi, M.; Koiwa, Y.; Fujii, H.; Fujita, K.; Maeda, A.; Kaneoka, Y., Hazama, S.; Nagano, H.; et al. Prediction of early recurrence of hepatocellular carcinoma after resection using digital pathology images assessed by machine learning. Modern Pathology 2021, 34 (2), 417-425. DOI: 10.1038/s41379-020-00671-z
(7) Herzfeld, A.; Mezl, V. A.; Knox, W. E. Enzymes metabolizing Δ1-pyrroline-5-carboxylate in rat tissues. Biochemical Journal 1977, 166 (1), 95-103. DOI: 10.1042/bj1660095 (accessed 1/13/2023).
(8) Ginguay, A.; Cynober, L.; Curis, E.; Nicolis, I. Ornithine Aminotransferase, an Important Glutamate-Metabolizing Enzyme at the Crossroads of Multiple Metabolic Pathways. Biology (Basel) 2017, 6 (1), 18. DOI: 10.3390/biology6010018 PubMed.
(9) Liu, W.; Hancock, C. N; Fischer, J. W.; Harman, M.; Phang, J M. Proline biosynthesis augments tumor cell growth and aerobic glycolysis: involvement of pyridine nucleotides. Scientific Reports 2015, 5 (1), 17206. DOI: 10.1038/srep17206. Phang, J. M. Proline Metabolism in Cell Regulation and Cancer Biology: Recent Advances and Hypotheses. Antioxidants & Redox Signaling 2017, 30 (4), 635-649. DOI: 10.1089/ars.2017.7350 (accessed 2023/01/13).
(10) Liu, W.; Le, A.; Hancock, C., Lane, A. N.; Dang, C. V.; Fan, T. W. M.; Phang, J. M. Reprogramming of proline and glutamine metabolism contributes to the proliferative and metabolic responses regulated by oncogenic transcription factor c-MYC. Proceedings of the National Academy of Sciences 2012, 109 (23), 8983-8988. DOI: 10.1073/pnas.1203244109 (accessed 2023/01/13).
(11) Peraino, C.; Bunville, L. G.; Tahmisian, T. N. Chemical, Physical, and Morphological Properties of Ornithine Aminotransferase from Rat Liver. Journal of Biological Chemistry 1969, 244 (9), 2241-2249. DOI: https://doi.org/10.1016/S0021-9258(19)78217-9.
(12) Phang, J. M.; Donald, S. P.; Pandhare, J.; Liu, Y. The metabolism of proline, a stress substrate, modulates carcinogenic pathways. Amino Acids 2008, 35 (4), 681-690 DOI: 10.1007/s00726-008-0063-4. Li, Y.; Bie, J.; Song, C.; Liu, M.; Luo, J. PYCR, a key enzyme in proline metabolism, functions in tumorigenesis. Amino Acids 2021, 53 (12), 1841-1850. DOI. 10.1007/s00726-021-03047-y.
(13) Shen, Y.-A.; Chen, C.-C.; Chen, B.-J.; Wu, Y.-T.; Juan, J.-R.; Chen, L.-Y.; Teng, Y.-C.; Wei, Y.-H. Potential Therapies Targeting Metabolic Pathways in Cancer Stem Cells. Cells 2021, 10 (7), 1772. DOI: 10.3390/cells10071772 PubMed.
(14) Burke, L.; Guterman, I.; Palacios Gallego, R.; Britton, R. G.; Burschowsky, D.; Tufarelli, C., Rufini, A. The Janus-like role of proline metabolism in cancer. Cell Death Discovery 2020, 6 (1), 104. DOI: 10.1038/s41420-020-00341-8.
(15) Tang, L; Zeng, J.; Geng, P.; Fang, C.; Wang, Y.; Sun, M.; Wang, C.; Wang, J.; Yin, P.; Hu, C.; et al. Global Metabolic Profiling Identifies a Pivotal Role of Proline and Hydroxyproline Metabolism in Supporting Hypoxic Response in Hepatocellular Carcinoma. Clinical Cancer Research 2018, 24 (2), 474-485. DOI: 10.1158/1078-0432.CCR-17-1707 (accessed 1/14/2023).
(16) Halama, A.; Suhre, K. Advancing Cancer Treatment by Targeting Glutamine Metabolism & mdash; A Roadmap. In Cancers, 2022; Vol. 14. Shanware, N. P.; Mullen, A. R.; DeBerardinis, R. J.; Abraham, R. T. Glutamine: pleiotropic roles in tumor growth and stress resistance. Journal of Molecular Medicine 2011, 89 (3), 229-236. DOI: 10.1007/s00109-011-0731-9. DeBerardinis, R. J.; Mancuso, A.; Daikhin, E.; Nissim, I.; Yudkoff, M.; Wehrli, S.; Thompson, C. B. Beyond aerobic glycolysis: Transformed cells can engage in glutamine metabolism that exceeds the requirement for protein and nucleotide synthesis. Proceedings of the National Academy of Sciences 2007, 104 (49), 19345-19350. DOI: 10.1073/pnas.0709747104 (accessed 2023/01/13).
(17) Cadoret, A., Ovejero, C.; Terris, B.; Souil, E.; Lévy, L.; Lamers, W. H.; Kitajewski, J.; Kahn, A.; Perret, C. New targets of β-catenin signaling in the liver are involved in the glutamine metabolism. Oncogene 2002, 21(54), 8293-8301. DO: 10.1038/sj.onc.1206118
(18) Colnot, S.; Decaens, T.; Niwa-Kawakita, M.; Godard, C.; Hamard, G.; Kahn, A.; Giovannini, M.; Perret, C. Liver-targeted disruption of Apc in mice activates β-catenin signaling and leads to hepatocellular carcinomas. Proceedings of the National Academy of Sciences 2004, 101 (49), 17216-17221. DOI: 10.1073/pnas.0404761101 (accessed 2023/01/13).
(19) Zigmond, E.; Ya'acov, A. B.; Lee, H.; Lichtenstein, Y.; Shalev, Z.; Smith, Y.; Zolotarov, L.; Ziv, E.; Kalman, R.; Le, H. V.; et al. Suppression of Hepatocellular Carcinoma by Inhibition of Overexpressed Ornithine Aminotransferase. ACS Medicinal Chemistry Letters 2015, 6 (8), 840-844. DOI: 10.1021/acsmedchemlett.5b00153.
(20) Liu, Y., Wu, L.; Li, K.; Liu, F.; Wang, L.; Zhang, D.; Zhou, J.; Ma, X.; Wang, S.; Yang, S. Ornithine aminotransferase promoted the proliferation and metastasis of non-small cell lung cancer via upregulation of miR-21. Journal of Cellular Physiology 2019, 234 (8), 12828-12838, https://doi.org/10.1002fjcp.27939. DOI: https://doi.org/10.1002/jcp.27939 (accessed 2023/01/13).
(21) Sun, Y.; Yang, J.; Cai, H.; Liu. J.; Liu, Y.; Luo. J.; Zhou, H. Differential OAT methylation correlates with cell infiltration in tumor microenvironment and overall survival postradiotherapy in oral squamous cell carcinoma patient. Journal of Oral Pathology & Medicine 2022, 51 (7), 611-619, https://doi.org/10.1111/jop.13328. DOI: https://doi.org/10.1111/jop.13328 (accessed 2023/01/13).
(22) Jiang, Z.; Wei, C.; Luo, Y.; Xiao, Y.; Wang, L.; Guo, W.; Yuan, X. Ornithine aminotransferase and carbamoyl phosphate synthetase 1 involved in ammonia metabolism serve as novel targets for early stages of gastric cancer. Journal of Clinical Laboratory Analysis 2022, 36 (10), e24692, https://doi.org/10.1002fjcla.24692. DOI: https://doi.org/10.1002/jcla.24692 (accessed 2023/01/13).
(23) John, R.; Fowler, L. Mammalian o-amino acid transaminases. P. Christen, & DE Metzler, Transaminases 1985, 376. Mehta, P. K.; Hale, T I; Christen, P. Aminotransferases: demonstration of homology and division into evolutionary subgroups. European Journal of Biochemistry 1993, 214 (2), 549-561, https.//doi.org/10.1111fj.1432-1033.1993.tb17953.x. DOI-https://doi.org/10.1111/j.1432-1033.1993.tb17953.x (accessed 2023/01/13).
(24) Markova, M., Peneff, C.; Hewlins, M. J. E.; Schirmer, T.; John, R. A. Determinants of Substrate Specificity in ω-Aminotransferases*. Journal of Biological Chemistry 2005, 280 (43), 36409-36416. DOI: https://doi.org/10.1074/jbc.M506977200.
(25) Storici, P.; Capitani, G.; Müller, R.; Schirmer, T.; Jansonius, J. N. Crystal structure of human ornithine aminotransferase complexed with the highly specific and potent inhibitor 5-fluoromethylornithine111 Edited by R. Huber. Journal of Molecular Biology 1999, 285 (1), 297-309. DOI: https://doi.org/10.1006/jmbi.1998.2289.
(26) Pan, Y.; Gerasimov, M. R.; Kvist, T.; Wellendorph, P.; Madsen, K. K.; Pera, E.; Lee, H.; Schousboe, A.; Chebib, M.; Brauner-Osborne, H.; et al. (1S, 3S)-3-amino-4-difluoromethylenyl-1-cyclopentanoic acid (CPP-115), a potent γ-aminobutyric acid aminotransferase inactivator for the treatment of cocaine addiction. J Med Chem 2012, 55 (1), 357-366. DOI: 10.1021/jm201231w PubMed. Lee, H.; Le, H. V.; Wu, R.; Doud, F.; Sanishvili, R.; Kellie, J. F.; Compton, P. D.; Pachaiyappan, B.; Liu, D.; Kelleher, N. L.; et al. Mechanism of Inactivation of GABA Aminotransferase by (E)- and (Z)-(1S,3S)-3-Amino-4-fluoromethylenyl-1-cyclopentanoic Acid. ACS Chem Biol 2015, 10 (9), 2087-2098. DOI: 10.1021/acschembio.5b00212 PubMed. Juncosa, J. I.; Takaya, K.; Le, H. V.; Moschitto, M. J.; Weerawarna, P. M.; Mascarenhas, R.; Liu, D.; Dewey. S. L.; Silverman. R. B. Design and Mechanism of (S)-3-Amino-4-(difluoromethylenyl)cyclopent-1-ene-1-carboxylic Acid, a Highly Potent γ-Aminobutyric Acid Aminotransferase Inactivator for the Treatment of Addiction. J Am Chem Soc 2018, 140 (6), 2151-2164. DOI: 10.1021/jacs.7b10965 PubMed.
(27) Zhu, W.; Butrin, A.; Melani, R. D.; Doubleday, P. F.; Ferreira, G. M.; Tavares, M. T.; Habeeb Mohammad, T. S.; Beaupre, B. A.; Kelleher, N. L.; Moran, G. R.; et al. Rational Design, Synthesis, and Mechanism of (3S,4R)-3-Amino-4-(difluoromethyl)cyclopent-1-ene-1-carboxylic Acid: Employing a Second-Deprotonation Strategy for Selectivity of Human Ornithine Aminotransferase over GABA Aminotransferase. J Am Chem Soc 2022, 144 (12), 5629-5642 DOI: 10.1021/jacs.2c00924. Zhu, W.; Doubleday, P. F.; Butrin, A.; Weerawama, P. M.; Melani, R. D.; Catlin, D. S.; Dwight, T. A.; Liu, D.; Kelleher, N. L.; Silverman, R. B. Remarkable and Unexpected Mechanism for (S)-3-Amino-4-(difluoromethylenyl)cyclohex-1-ene-1-carboxylic Acid as a Selective Inactivator of Human Ornithine Aminotransferase. J Am Chem Soc 2021, 143 (21), 8193-8207. DOI. 10.1021/jacs.1c03572. Zhu, W.; Doubleday, P. F.; Catlin, D. S.; Weerawarna, P. M.; Butrin, A.; Shen, S.; Wawrzak, Z.; Kelleher, N. L.; Liu, D.; Silverman, R. B. A Remarkable Difference That One Fluorine Atom Confers on the Mechanisms of Inactivation of Human Ornithine Aminotransferase by Two Cyclohexene Analogues of γ-Aminobutyric Acid. J Am Chem Soc 2020, 142 (10), 4892-4903. DOI: 10.1021/jacs.0c00193. Shen, S.; Butrin, A.; Doubleday, P. F.; Melani, R. D.; Beaupre, B. A.; Tavares, M. T.; Ferreira, G. M.; Kelleher, N. L.; Moran, G. R.; Liu, D.; et al. Turnover and Inactivation Mechanisms for (S)-3-Amino-4,4-difluorocyclopent-1-enecarboxylic Acid, a Selective Mechanism-Based Inactivator of Human Ornithine Aminotransferase. J Am Chem Soc 2021, 143 (23), 8689-8703. DOI: 10.1021/jacs.1c02456 PubMed.
(28) Butrin, A.; Butrin, A.; Wawrzak, Z.; Moran, G. R.; Liu, D. Determination of the pH dependence, substrate specificity, and turnovers of alternative substrates for human ornithine aminotransferase. J Biol Chem 2022, 298 (6), 101969. DOI: 10.1016/j.jbc.2022.101969.
(29) Danielson, M. A., Falke, J. J. Use of 19F NMR to probe protein structure and conformational changes. Annu Rev Biophys Biomol Struct 1996, 25, 163-195. DOI: 10.1146/annurev.bb.25.060196.001115.
(30) Martinez, D.; Gerig, J. T. Intermolecular (1)H[(19)F] NOEs in studies of fluoroalcohol-induced conformations of peptides and proteins. J Magn Reson 2001, 152 (2), 269-275. DOI: 10.1006/jmre.2001.2403.
(31) Bissantz, C.; Kuhn, B.; Stahl, M. A medicinal chemist's guide to molecular interactions. J Med Chem 2010, 53 (14), 5061-5084. DOI: 10.1021fjm100112j.
(32) Pham, T. V.; Murkin, A. S.; Moynihan, M. M.; Harris, L.; Tyler, P. C.; Shetty, N.; Sacchettini, J. C.; Huang, H. L.; Meek, T. D. Mechanism-based inactivator of isocitrate lyases 1 and 2 from Mycobacterium tuberculosis. Proc Natl Acad Sci USA 2017, 114 (29), 7617-7622. DOI: 10.1073/pnas.1706134114.
(33) Frieden, E.; Walter, C. Prevalence and significance of the product inhibition of enzymes. Nature 1963, 198, 834-837. DOI: 10.1038/198834a0.
(34) Lee, H.; Doud, E. H.; Wu, R.; Sanishvili, R.; Juncosa, J. I.; Liu, D.; Kelleher, N. L.; Silverman, R. B. Mechanism of inactivation of gamma-aminobutyric acid aminotransferase by (1S,3S)-3-amino-4-difluoromethylene-1-cyclopentanoic acid (CPP-115). J Am Chem Soc 2015, 137 (7), 2628-2640. DOI: 10.1021/ja512299n.
(35) Shen, S.; Butrin, A.; Doubleday, P. F., Melani, R. D.; Beaupre, B. A.; Tavares, M. T.; Ferreira, G. M.; Kelleher, N. L.; Moran, G. R.; Liu, D.; et al. Turnover and Inactivation Mechanisms for (S)-3-Amino-4,4-difluorocyclopent-1-enecarboxylic Acid, a Selective Mechanism-Based Inactivator of Human Ornithine Aminotransferase. J Am Chem Soc 2021, 143 (23), 8689-8703. DOI: 10.1021/jacs.1c02456
(36) Wheless, J. W.; Ramsay, R. E.; Collins, S. D. Vigabatrin. Neurotherapeutics 2007, 4 (1), 163-172. DOI: 10.1016/j.nurt.2006.11.008.
(37) Mascarenhas, R.; Le, H. V.; Clevenger, K. D.; Lehrer, H. J.; Ringe, D.; Kelleher, N. L.; Silverman, R. B.; Liu, D. Selective Targeting by a Mechanism-Based Inactivator against Pyridoxal 5′-Phosphate-Dependent Enzymes: Mechanisms of Inactivation and Alternative Turnover. Biochemistry 2017, 56 (37), 4951-4961. DOI: 10.1021/acs.biochem.7b00499.
(38) Moschitto, M. J.; Doubleday, P. F., Catlin, D. S.; Kelleher, N. L., Liu, D.; Silverman, R. B. Mechanism of Inactivation of Ornithine Aminotransferase by (1S,3S)-3-Amino-4-(hexafluoropropan-2-ylidenyl)cyclopentane-1-carboxylic Acid. J Am Chem Soc 2019, 141 (27), 10711-10721. DOI: 10.1021/jacs.9b03254.
(39) Heath, T. K.; Lutz, M. R., Jr.; Reidi, C. T., Guzman, E. R., Herbert, C. A., Nocek, B. P.; Holz, R. C.; Olsen. K. W.; Ballicora, M. A.; Becker, D. P. Practical spectrophotometric assay for the dapE-encoded N-succinyl-L,L-diaminopimelic acid desuccinylase, a potential antibiotic target. PLoS One 2018, 13 (4), e0196010. DOI: 10.1371/journal.pone.0196010.
(40) Vilar, S.; Cozza, G.; Moro, S Medicinal chemistry and the molecular operating environment (MOE): application of QSAR and molecular docking to drug discovery. Curr Top Med Chem 2008, 8 (18), 1555-1572. DOI: 10.2174/156802608786786624.

In accordance with this disclosure, various other compounds, varied structurally, stereochemically and/or configurationally, are available through such incorporated synthetic procedures and techniques or straight-forward modifications thereof, such modifications as would also be known and understood by those skilled in the art and made aware of this invention, such procedures, techniques and modifications limited only by the commercial or synthetic availability of any corresponding reagent or starting material.

1. A compound of the following formula or a dissociated form, a non-protonated form, a zwitterion form, or a salt thereof: 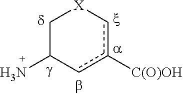
wherein
a double bond is present between the α and ζ carbons, or between the α and β carbons, or not present between the α and ζ carbons and the α and β carbons;
X is CR¹R² or C═CR¹R²; and
R¹ and R² are independently hydrogen or halo, providing at least one of R¹ and R² is halo.

2. The compound of claim 1, wherein the double bond is between the α and ζ carbons.

3. The compound of claim 1, wherein the double bond is between the α and β carbons.

4. The compound of claim 1, wherein the double bond is not present between the α and ζ carbons and the α and β carbons.

5. The compound of any one of claims 1-4, wherein X is CR¹R².

6. The compound of any one of claims 1-4, wherein X is C═CR¹R².

7. The compound of any one of claims 1-6, wherein at least one of R¹ and R² is F.

8. The compound of any one of claims 1-6, wherein R¹ and R² are F.

9. The compound of any one of claims 1-8, wherein the compound is a salt comprising a substituent that is an ammonium substituent or a carboxylate substituent.

10. The compound of claim 9, wherein the ammonium substituent has a counter ion that is a conjugate base of a protic acid.

11. The compound of any one of claims 1-8 in zwitterion form comprising an ammonium moiety and a carboxylate moiety.

12. The compound of claim 1 of a formula selected from the group consisting of:

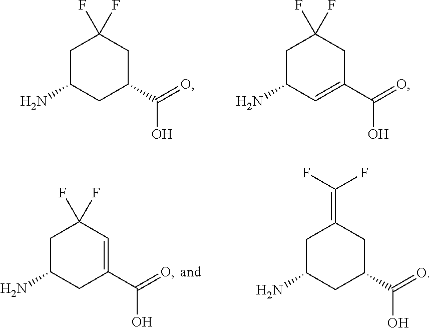

13. The compound of claim 1 of a formula that is:

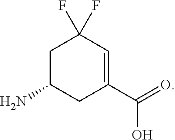 [SMILES: N[C@H]1CC(C(=O)O)=CC(F)(F)C1]

14. The compound of claim 1 of a formula that is:

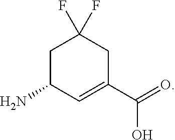 [SMILES: N[C@H]1C=C(C(=O)O)CC(F)(F)C1]

15. A pharmaceutical composition comprising the compound of any one of claims 1-14 and a pharmaceutically suitable carrier, diluent, or excipient.

16. A method of modulating ornithine aminotransferase (OAT) activity, the method comprising contacting the compound of any one of claims 1-14, or the pharmaceutical composition of claim 15, with a medium comprising an OAT, wherein the compound is present in an amount sufficient to modulate OAT activity.

17. The method of claim 16, wherein the contact is in vivo.

18. The method of claim 16 or 17, wherein the OAT is human ornithine aminotransferase (hOAT).

19. A method of reducing activity of an OAT expressed by a human cancer, the method comprising contacting the compound of any one of claims 1-14, or the pharmaceutical composition of claim 15, with the cancer expressing an OAT, wherein the compound is present in an amount that is effective to reduce OAT activity.

20. The method of claim 19, wherein the contact is in vivo.

21. The method of claim 19 or 20, wherein the contact is within a human subject in need of a treatment for cancer.

22. The method of any one of claims 19-21, wherein the OAT is human ornithine aminotransferase (hOAT).

23. A method for treating cancer in a subject in need thereof, the method comprising administering to the subject a therapeutically effective amount of the compound of any one of claims 1-14, or the pharmaceutical composition of claim 15.

24. The method of claim 23, wherein the cancer is characterized by expression or overexpression of human ornithine aminotransferase (hOAT).

25. The method of claim 23 or 24, wherein the cancer is hepatocellular carcinoma (HCC).
